(19) United States
(12) Patent Application Publication
(10) Pub. No.: US 2024/0299167 A1
(43) Pub. Date: Sep. 12, 2024

(54) DELIVERY APPARATUS AND METHODS FOR IMPLANTING PROSTHETIC HEART VALVES

(71) Applicant: EDWARDS LIFESCIENCES CORPORATION, Irvine, CA (US)

(72) Inventors: Elazar Levi Schwarcz, Netanya (IL); Ofir Witzman, Harish (IL); Eitan Atias, Netanya (IL); Natanel Simcha Sirote, Zikhron Ya’akov (IL); Noam Miller, Givatayim (IL); Aviran Pitusi, Rosh Haayin (IL)

(21) Appl. No.: 18/664,921

(22) Filed: May 15, 2024

Related U.S. Application Data
(63) Continuation of application No. PCT/US2022/050710, filed on Nov. 22, 2022.
(60) Provisional application No. 63/420,166, filed on Oct. 28, 2022.
(60) Provisional application No. 63/282,463, filed on Nov. 23, 2021.

(51) Int. Cl. A61F 2/24

(52) U.S. Cl. CPC A61F 2/243

(57) ABSTRACT

A delivery apparatus for a prosthetic heart valve includes a handle, one or more actuator drivers, and a gearbox disposed within the handle and coupled to rotate the actuator drivers relative to the handle. The gearbox can include a counter-rotation gear train that can be operated to rotate two sets of actuator drivers in opposite directions. One or more of the actuator drivers can have an associated torque limiter that prevents overloading of the actuator driver. The gearbox can be pivotably mounted within the handle. The gearbox can be configured to engage a stop member within the handle to limit pivoting of the gearbox in a predetermined direction. The handle can include a sensor that is positioned to measure torque applied to the prosthetic heart valve while rotating the actuation drivers.

### CROSS-REFERENCE TO RELATED APPLICATIONS

This application is a continuation of International Patent Application No. PCT/US2022/050710, filed Nov. 22, 2022, which claims priority to U.S. Provisional Application Nos. 63/420,166, filed Oct. 28, 2022, and 63/282,463, filed Nov. 23, 2021. The prior applications are incorporated by reference herein in their entirety.

### FIELD

The field relates to implantable prosthetic devices, such as prosthetic heart valves, and to delivery apparatus and methods for implanting prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable.

In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Prosthetic heart valves that rely on a mechanical actuator for expansion can be referred to as “mechanically expandable” prosthetic heart valves. Mechanically expandable prosthetic heart valves can provide one or more advantages over self-expandable and balloon-expandable prosthetic heart valves. For example, mechanically expandable prosthetic heart valves can be expanded to various diameters. Mechanically expandable prosthetic heart valves can also be compressed after an initial expansion (for example, for repositioning and/or retrieval).

### SUMMARY

Described herein are delivery apparatus and methods for implanting prosthetic heart valves. The disclosed delivery apparatus and methods can, for example, reduce the difficulty and/or the time needed to implant a prosthetic heart valve. The disclosed delivery apparatus are relatively simple and easy to use and include various safeguards, which can help to ensure that the prosthetic heart valve is safely and securely implanted.

A delivery apparatus for a prosthetic heart valve can include a handle and a shaft assembly coupled to the handle. The delivery apparatus can further include one or more actuation assemblies that can be used to releasably couple the prosthetic heart valve to the shaft assembly and to radially expand and/or compress the prosthetic heart valve.

In some examples, a delivery apparatus for a prosthetic heart valve can be summarized as including a handle having a proximal end, a distal end, and a cavity extending from the proximal end to the distal end; a first actuator driver having a proximal end portion disposed within the cavity and a distal end portion extending out of the cavity; a second actuator driver having a proximal end portion disposed within the cavity and a distal end portion extending out of the cavity; and a gear train disposed within the cavity and coupled to the proximal end portions of the first and second actuator drivers, the gear train configured to simultaneously rotate the first and second actuator drivers in opposite directions.

In some examples, a delivery apparatus for a prosthetic heart valve can be summarized as including a handle having a longitudinal axis and a cavity extending along the longitudinal axis; a set of first actuator drivers, each first actuator driver having a proximal end portion disposed within the cavity and a distal end portion extending out of the cavity; a set of second actuator drivers, each second actuator driver having a proximal end portion disposed within the cavity and a distal end portion extending out of the cavity; a first driving gear coupled to the first actuation drivers, the first driving gear configured to rotate the first actuator drivers in a first direction; and a second driving gear coupled to the second actuator drivers, the second driving gear configured to rotate the second actuator drivers in a second direction that is opposite to the first direction.

In some examples, a prosthetic heart valve can be summarized as including a frame having an inflow end, an outflow end, and a longitudinal axis extending from the inflow end to the outflow end, the frame movable between a radially expanded configuration and a radially compressed configuration; a first actuator coupled to the frame at a first position; and a second actuator coupled to the frame at a second position that is spaced from the first position along a circumference of the frame. Rotation of the first actuator in a first rotational direction and rotation of the second actuator in a second rotational direction, opposite to the first rotational direction, moves the frame between the radially expanded configuration and the radially compressed configuration.

In some examples, a delivery assembly can be summarized as including a prosthetic heart valve comprising a frame having an inflow end, an outflow end, and a longitudinal axis extending from the inflow end to the outflow end, the frame movable between a radially expanded configuration and a radially compressed configuration; a first actuator coupled to the frame at a first position; and a second actuator coupled to the frame at a second position that is spaced from the first position along a circumference of the frame. The delivery assembly comprises a handle having a proximal end, a distal end, and a cavity extending from the proximal end to the distal end; a first actuator driver having a proximal end portion disposed within the cavity and a distal end portion extending out of the cavity and releasably coupled to the first actuator; a second actuator driver having a proximal end portion disposed within the cavity and a distal end portion extending out of the cavity and releasably coupled to the second actuator; and a gear train disposed within the cavity and coupled to the proximal end portions of the first and second actuator drivers, the gear train configured to simultaneously rotate the first and second actuator drivers in opposite directions.

In some examples, a delivery apparatus for a prosthetic heart valve can be summarized as including a handle having a cavity; a gearbox disposed within the cavity, the gearbox comprising at least one output shaft and a gear coupled thereto; an actuator driver having a predefined torque limit range; and a rotatable assembly coupling the at least one output shaft to the actuator driver, the rotatable assembly having a first rotational state wherein the at least one output shaft and the actuator driver rotate together about a longitudinal axis and a second rotational state wherein the at least one output shaft and the actuator driver do not rotate together about the longitudinal axis. The first rotational position corresponds to when a torque applied to the actuator driver is below the predefined torque limit range, and the second rotational position corresponds to when the torque applied to the actuator driver is within the predefine torque limit range.

In some examples, a delivery apparatus for a prosthetic heart valve can be summarized as including a handle having a cavity; a gearbox disposed within the cavity, the gearbox comprising a plurality of output shafts and a plurality of output gears coupled thereto; a plurality of actuator drivers, each actuator driver having a predefined torque limit range; and a plurality of rotatable assemblies, each rotatable assembly coupled at a first end to one of the output shafts and at a second end to one of the actuator drivers. Each rotatable assembly comprises a first rotatable body; a second rotatable body; and a rotational biasing member coupling the first rotatable body to the second rotatably body. The rotational biasing member biases the first rotatable body and the second rotatable body to a position in which the first rotatable body and the second rotatable body rotate together about a longitudinal axis when a torque applied to the actuator driver is below the predefined torque limit range. The rotational biasing member permits relative rotation between the first rotatable body and the second rotatable body about the longitudinal axis when the torque applied to the actuator driver is within the predefined torque limit range.

In some examples, a delivery apparatus for a prosthetic heart valve can be summarized as including a handle body having a longitudinal axis; a gearbox pivotable mounted within the handle body and about the longitudinal axis; and a stop member coupled to the handle body and positioned to limit pivoting of the gearbox about the longitudinal axis when the gearbox is pivoted in a predetermined direction.

In some examples, a delivery apparatus for a prosthetic heart valve can be summarized as including a handle body having a longitudinal axis; a load cell coupled to the handle body, the load cell having a first axial axis positioned tangentially to a circular path centered around the longitudinal axis; and a gearbox pivotably mounted about the longitudinal axis, the gearbox having a protrusion member with a first axial axis positioned tangentially to the circular path, the protrusion member configured to contact the load cell as the gearbox is pivoted in a predetermined direction corresponding to operation of the gearbox to expand the prosthetic heart valve.

In some examples, a delivery apparatus for a prosthetic heart valve can be summarized as including an actuator driver; a gearbox comprising at least one output shaft; an engagement member coupled to the at least one output shaft and rotatable about a longitudinal axis with the at least one output shaft, the engagement member having a first engagement surface and a first locking surface spaced apart along the longitudinal axis; a driver member coupled to the actuator driver and rotatable about the longitudinal axis, the driver member having a second engagement surface in opposing relation to the first engagement surface and engaged with the first engagement surface; and a base member rotationally fixed relative to the longitudinal axis, the base member having a second locking surface in opposing relation to the first locking surface; wherein the engagement member is axially displaceable along the longitudinal axis in response to a torque on the actuator driver and between a first position in which the first locking surface is separated from the second locking surface and a second position in which the first locking surface is interlocked with the second locking surface, and wherein the second position corresponds to a state in which the torque on the actuator driver exceeds a threshold.

In some examples, a delivery apparatus for a prosthetic heart valve can be summarized as including a handle having a cavity; a gearbox disposed within the cavity, the gearbox comprising at least one output shaft; an actuator driver extending into the cavity; and a torque limiter coupling the actuator driver to the at least one output shaft, the torque limiter comprising: an engagement member coupled to the at least one output shaft and rotatable about a longitudinal axis in response to rotation of the at least one output shaft, the engagement member comprising a set of engagement teeth at a first end and a first set of locking teeth at a second end spaced from the first end; a driver member rotatable about the longitudinal axis and coupled to the actuator driver, the driver member comprising a set of driver teeth in opposing relation to the set of engagement teeth and slidably engaged with the set of engagement teeth; a base member rotationally fixed relative to the longitudinal axis, the base member comprising a second set of locking teeth in opposing relation to the first set of locking set; and wherein the engagement member is axially displaceable along the longitudinal axis in response to a torque on the actuator driver, and wherein the engagement member is axially displaceable to engage the first set of locking teeth with the second set of locking teeth when the torque on the actuator driver exceeds a threshold.

In some examples, a method can be summarized as including coupling a prosthetic heart valve to at least one actuator driver of a delivery apparatus, wherein an engagement member is movably coupled to the at least one actuator driver and fixedly coupled to an output shaft of a gearbox of the delivery apparatus, wherein the engagement member is axially displaceable along a longitudinal axis and between the at least one actuator driver and a base member that is rotationally fixed relative to the longitudinal axis in response to a torque on the at least one actuator driver; and rotating the output shaft of the gearbox to rotate the at least one actuator driver in a first direction to radially expand the prosthetic heart valve to a working diameter, wherein rotation of the output shaft automatically stops by engagement of the engagement member with the base member when the torque on the at least one actuator driver exceeds a threshold.

### BRIEF DESCRIPTION OF THE DRAWINGS

FIG. 1 is a perspective view of a prosthetic heart valve.

FIG. 2A is a perspective view of the prosthetic heart valve in a radially expanded configuration with the valvular structure removed, depicting actuator heads at an outflow end of the frame.

FIG. 2B is a perspective view of the prosthetic heart valve in a radially expanded configuration, depicting actuator heads at an inflow end of the frame.

FIG. 3 is a detail view of an actuator of the prosthetic heart valve.

FIG. 4A is a side view of a proximal end portion of a delivery apparatus.

FIG. 4B is a side view of a distal end portion of a delivery apparatus with the prosthetic heart valve in a radially expanded configuration coupled thereto.

FIG. 5 is a cross-sectional view of a shaft assembly of the delivery apparatus, taken along line 5-5 of FIG. 4B.

FIG. 6 is a perspective view of a portion of an actuation assembly of the delivery apparatus.

FIG. 7A is a perspective view of the actuation assembly of the delivery apparatus aligned with an actuator of the prosthetic heart valve.

FIG. 7B is a perspective view of the actuation assembly engaged with the actuator.

FIG. 7C is a perspective view of the outer sleeve of the actuation assembly engaged with the frame of the prosthetic heart valve.

FIG. 8 is a cross-section of a handle of the delivery apparatus, taken along line 8-8 of FIG. 4A.

FIG. 9A is a portion of the handle of the delivery apparatus depicting a gearbox within the handle coupled to a knob of the handle.

FIG. 9B is a perspective view of the gearbox with the gearbox housing depicted as transparent.

FIG. 9C is a perspective view of a portion of the handle of the delivery apparatus depicting compartments inside the gearbox housing.

FIG. 10A is a perspective view of a gear train of the gearbox.

FIG. 10B is a plan view of the gear train in a direction parallel to the longitudinal axis of the handle.

FIG. 10C is a plan view of the gear train in a direction transverse to the longitudinal axis of the handle.

FIG. 11 is another perspective view of the prosthetic heart valve without the valvular structure and illustrating division of the actuation rods into two sets.

FIG. 12 is a schematic of a delivery assembly including the prosthetic heart valve in a radially expanded configuration and the delivery apparatus,

FIG. 13A is a perspective view of a torque limiter for an actuator driver.

FIG. 13B is a cross-sectional view of the torque limiter along lines 13B-13B.

FIG. 14 is a perspective view of a torsion spring.

FIG. 15 is a perspective view of a first rotational body of a rotational assembly of the torque limiter.

FIG. 16 is a perspective view of a second rotational body of the rotational assembly of the torque limiter.

FIG. 17A is a cross-sectional view of the torque limiter generally along the line 17A-17A as depicted in FIG. 13B.

FIG. 17B is a cross-sectional view of the torque limiter generally along the line 17B-17B as depicted in FIG. 13B.

FIG. 18 is a cross-sectional view of the torque limiter disposed within a housing.

FIG. 19 is a cross-sectional view of the torque limiter within a housing taken along the line 19-19 as depicted in FIG. 18.

FIGS. 20A and 20B illustrate approximation of the arms of the torsion spring during twisting of the torsion spring.

FIG. 21A is a cross-sectional view of a proximal end portion of the handle depicting a load cell mounted to the body of the handle.

FIG. 21B is a portion of the handle depicting a plate extension on the gearbox in contact with the load cell.

FIG. 22A is a top view of the gearbox housing.

FIG. 22B is a side view of the gearbox housing.

FIG. 22C is a proximal end view of the gearbox housing.

FIG. 22D is a cross-sectional view of the gearbox housing along line 22D-22D as depicted in FIG. 22B.

FIG. 22E is a cross-sectional view of the gearbox housing along line 22E-22E as depicted in FIG. 22B.

FIG. 22F is a cross-sectional view of the gearbox housing along line 22F-22F as depicted in FIG. 22B.

FIG. 22G is a cross-sectional view of the gearbox housing along line 22G-22G as depicted in FIG. 22B.

FIG. 22H is a cross-sectional view of the gearbox housing along line 22H-22H as depicted in FIG. 22B.

FIG. 22I is a perspective view of a distal end portion of the gearbox housing.

FIG. 22J is a distal end view of the gearbox housing.

FIG. 22K is a perspective view of the gearbox illustrating an encoder mounted on an output shaft.

FIG. 23 is a perspective view of a portion of the handle depicting a pull body coupled to a knob and the gearbox.

FIG. 24A is a perspective view of a portion of the handle depicting a pull body coupled to the gearbox.

FIG. 24B is a perspective view of the pull body viewed from an end of the pull body.

FIG. 24C is a perspective view of the pull body.

FIG. 24D is a cross-sectional view of the pull body along a plane extending along line 24D-24D as depicted in FIG. 24C.

FIG. 24E is a cross-sectional view a portion of the handle along a plane extending along line 24E-24E as depicted in FIG. 24A.

FIG. 24F is a cross-sectional view of a portion of the handle along a plane extending along line 24F-24F as depicted in FIG. 24A.

FIG. 25A is a cross-sectional of a knob having inner channels.

FIG. 25B is a cross-sectional view of the knob along a plane extending along line 25B-25B as depicted in FIG. 25A.

FIG. 26 is a cross-sectional view of the handle along line 26-26 as depicted in FIG. 4A.

FIG. 27A is a cross-sectional view of a torque limiter for an actuator driver, according to some examples, in a locked state.

FIG. 27B is a cross-sectional view of the torque limiter of FIG. 27A in an unlocked state.

FIG. 28A is a perspective view of a driver member of the torque limiter of FIGS. 27A and 27B.

FIG. 28B is a perspective view of an engagement member of the torque limiter of FIGS. 27A and 27B.

FIG. 28C is a schematic diagram of forces between opposing tooth surfaces of the driver member of FIG. 28A and the engagement member of FIG. 28B.

FIG. 29A is a perspective view of a torque limiter for an actuator driver, according to some examples.

FIG. 29B is a detail view illustrating the geometry of the teeth of the engagement and driver members of the torque limiter of FIG. 29A.

FIG. 29C is a cross-sectional view of a torque limiter along line 29C-29C as depicted in FIG. 29A.

FIGS. 30A-30D illustrate a sequence of states of the torque limiter of FIG. 29A during transition from an unlocked state to a locked state.

### DETAILED DESCRIPTION

### General Considerations

The subject matter is described with implementations and examples. In some cases, as will be recognized by one skilled in the art, the disclosed implementations and examples may be practiced without one or more of the disclosed specific details, or may be practiced with other methods, structures, and materials not specifically disclosed herein. All the implementations and examples described herein and shown in the drawings may be combined without any restrictions to form any number of combinations, unless the context clearly dictates otherwise, such as if the proposed combination involves elements that are incompatible or mutually exclusive. The sequential order of the acts in any process described herein may be rearranged, unless the context clearly dictates otherwise, such as if one act requires the result of another act as input.

In the interest of conciseness, and for the sake of continuity in the description, same or similar reference characters may be used for same or similar elements in different figures, and description of an element in one figure will be deemed to carry over when the element appears in other figures with the same or similar reference character. In some cases, the term “corresponding to” may be used to describe correspondence between elements of different figures. In an example usage, when an element in a first figure is described as corresponding to another element in a second figure, the element in the first figure is deemed to have the characteristics of the other element in the second figure, and vice versa, unless stated otherwise.

The word “comprise” and derivatives thereof, such as “comprises” and “comprising”, are to be construed in an open, inclusive sense, that is, as “including, but not limited to”. The singular forms “a”, “an”, “at least one”, and “the” include plural referents, unless the context dictates otherwise. The term “and/or”, when used between the last two elements of a list of elements, means any one or more of the listed elements. The term “or” is generally employed in its broadest sense, that is, as meaning “and/or”, unless the context clearly dictates otherwise.

The term “coupled” without a qualifier generally means physically coupled or linked and does not exclude the presence of intermediate elements between the coupled elements absent specific contrary language. The term “plurality” or “plural” when used together with an element means two or more of the element. Directions and other relative references (for example, inner and outer, upper and lower, above and below, left and right, and proximal and distal) may be used to facilitate discussion of the drawings and principles herein but are not intended to be limiting.

The terms “proximal” and “distal” are defined relative to the use position of a delivery apparatus. In general, the end of the delivery apparatus closest to the user of the apparatus is the proximal end, and the end of the delivery apparatus farthest from the user (for example, the end that is inserted into a patient's body) is the distal end. The term “proximal” when used with two spatially separated positions or parts of an object can be understood to mean closer to or oriented towards the proximal end of the delivery apparatus. The term “distal” when used with two spatially separated positions or parts of an object can be understood to mean closer to or oriented towards the distal end of the delivery apparatus.

### Intro to the Disclosed Technology

Described herein are prosthetic heart valves, delivery apparatus, and methods for implanting prosthetic heart valves. The prosthetic heart valves can include two or more actuators that can be operated to radially expand or radially compress the prosthetic heart valve. The delivery apparatus can include actuator drivers to releasably engage and operate the actuators.

In some examples, the delivery apparatus can include a counter-rotation mechanism operatively coupled to the actuators such that a net moment force on the prosthetic heart valve while operating the actuators is substantially zero. During expansion of the prosthetic heart valve using the actuators, the counter-rotation movement of the actuators can help maintain the prosthetic heart valve at a rotationally fixed position relative to the native anatomy.

In some examples, the counter-rotation mechanism can include a gearbox pivotably mounted within a handle of the delivery apparatus and coupled to the actuator drivers. In some examples, a stop member can be arranged within the handle to engage and limit pivoting of the gearbox during expansion of the prosthetic heart valve. In some examples, the stop member can include a sensor to measure load on the gearbox while the gearbox is engaged with the stop member.

In some examples, the delivery apparatus can include a mechanism that limits the torque applied to an actuator driver during expansion of the prosthetic heart valve. The torque limiter can be configured to halt a gear train of the gearbox once the torque applied to the actuator driver is within a tolerance of a predetermined maximum torque.

### Examples of the Disclosed Technology

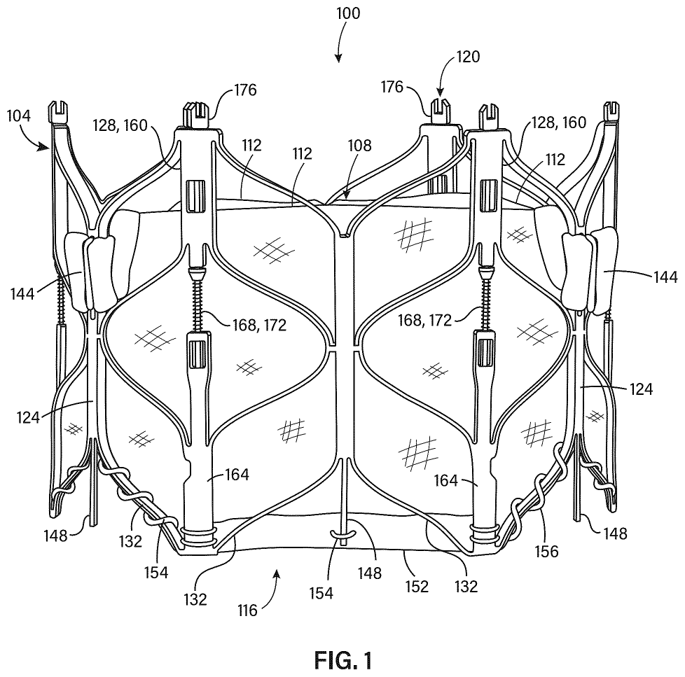

FIG. 1 illustrates a prosthetic heart valve 100, according to some examples. The prosthetic heart valve 100 can be configured to replace a native heart valve (for example, aortic, mitral, pulmonary, and/or tricuspid valves). The prosthetic heart valve 100 is illustrated as a mechanically expandable prosthetic heart valve that can be radially compressed for delivery to an implantation location within a patient's body and then radially expanded to a working diameter at the implantation location. The prosthetic heart valve 100 can include a frame 104 having an annular shape. The prosthetic heart valve 100 can further include a valvular structure 108 supported within and coupled to the frame 104.

In the example, the valvular structure 108 includes one or more leaflets 112 made of flexible material and configured to open and close to regulate blood flow. In some examples, the valvular structure 108 can have three leaflets 112, which can be arranged to collapse in a tricuspid arrangement. The leaflets 112 can be made in whole or in part from pericardial tissue (for example, bovine pericardial tissue), biocompatible synthetic materials, or various other suitable natural or synthetic materials.

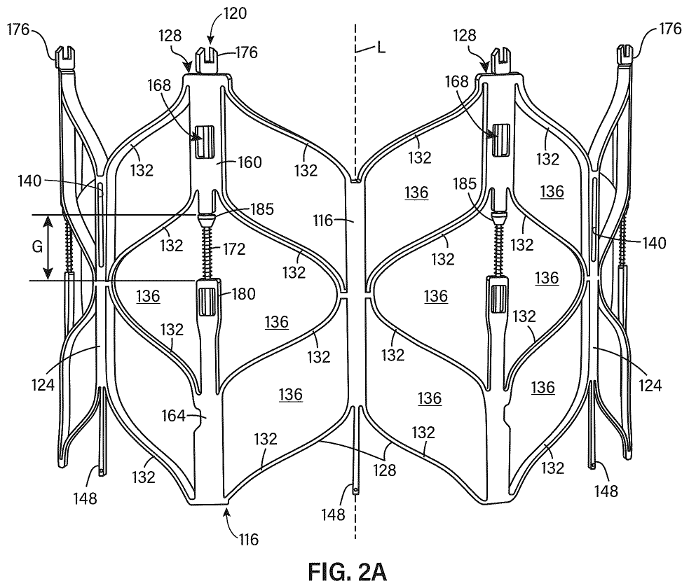

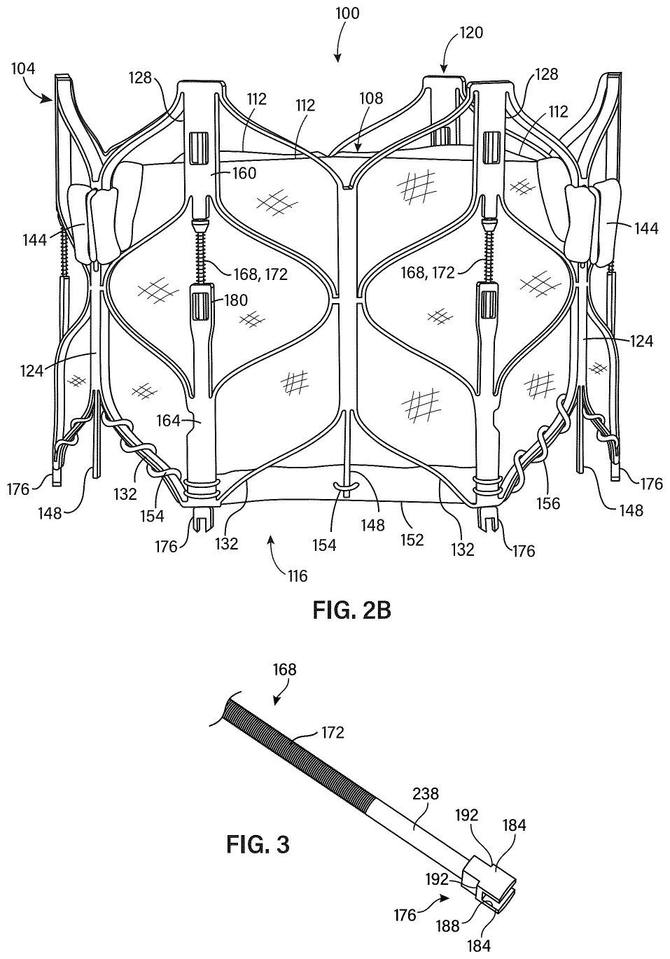

As illustrated more clearly in FIG. 2A, the frame 104 has an inflow end 116, an outflow end 120, and a longitudinal axis L extending in a direction from the inflow end 116 to the outflow end 120. The frame 104 can include a plurality of support posts 124, 128 aligned with the longitudinal axis L and spaced along a circumference of the frame 104. In some examples, the support posts 124, 128 can be arranged in an alternating manner along the circumference of the frame 104. The frame 104 can further include a plurality of struts 132 extending circumferentially between adjacent support posts 124, 128 and interconnecting the support posts 124, 128. The struts 132 and support posts 124, 128 define cells 136 of the frame 104. As illustrated, the struts 132 can have a curved shape.

As illustrated in FIGS. 1 and 2A, one or more commissure windows 140 can be formed in one or more of the support posts 124. Commissures 144 can be formed at the commissure windows 140 to couple the leaflets 112 to the frame 104. One or more of the support posts 124 can further include cantilevered struts 148 extending to the inflow end 116 of the frame 104. In some cases, inflow edge portions 152 of the leaflets 112 can be attached to the cantilevered struts 148 (for example, by sutures 154) and/or to selected struts 132 of the frame 104 (for example, using sutures 156).

In some examples, the frame 104 can be adjusted between a radially expanded configuration and a radially compressed configuration by deflecting the struts 132. In some examples, the frame 104 (for example, the posts and struts) can be made of biocompatible plastically-expandable materials that will allow the frame 104 to be adjusted between the radially expanded configuration and radially compressed configuration. Suitable examples of plastically-expandable materials that can be used in forming the frame 104 include, but are not limited to, stainless steel, cobalt chromium alloy, and/or nickel titanium alloy (which can also be referred to as “NiTi” or “nitinol”).

Referring to FIG. 2A, in some examples, one or more actuators 168 can be coupled to the support posts 128 and used to adjust the frame 104 between the radially expanded configuration and the radially compressed configuration. In some examples, each support post 128 can include an upper post member 160 and a lower post member 164 (the terms “upper” and “lower” are relative to the orientation of the frame 104 in FIG. 1) aligned with the longitudinal axis L of the frame 104 and having opposing ends separated by a gap G. The respective actuator 168 can be coupled to the post members 160, 164 and operable to increase or decrease the gap G in order to radially compress or expand the frame 104.

In some examples, the actuator 168 can include an actuator rod 172 with an attached actuator head 176. In the examples illustrated in FIGS. 2A and 2B, the actuator rod 172 extends through or into the post members 160, 164 and across the gap G. In the example illustrated in FIG. 2A, the actuator rod 172 is inserted into the upper post member 160 from the outflow end 120, and the actuator head 176 is disposed or retained at the outflow apex of the upper post member 160. In the example illustrated in FIG. 2B, the actuator rod 172 is inserted into the lower post member 164 from the inflow end 116, and the actuator head 176 is disposed or retained at the inflow apex of the lower post member 164.

In some examples, the actuator rod 172 is externally threaded. As illustrated in FIGS. 2A and 2B, the lower post member 164 can include a nut 180 with an internal thread to threadedly engage the actuator rod 172. In this case, the actuator rod 172 can be translated in a longitudinal direction by rotating the actuator rod 172 relative to the nut 180. In some examples, the actuator rod 172 can be freely slidable relative to the upper post member 160. In other examples, the actuator rod 172 can threadedly engage the upper post member 160.

As illustrated in FIG. 3, the actuator head 176 can include a pair of protrusions 184 forming a slot 188. The actuator head 176 can further include one or more shoulders 192. As further described in greater detail herein, an actuation assembly of the delivery apparatus can releasably engage the actuator head 176 via the slot 188 and shoulders 192.

Referring to FIGS. 2A and 2B, in one scenario, the actuator rod 172 can be rotated in a first direction to move the upper post member 160 towards the lower post member 164 and thereby decrease the size of gap G, which can have the effect of radially expanding the frame 104. In another scenario, the lower post member 164 may be held steady while the actuator rod 172 is rotated in a second direction to move the upper post member 160 away from the lower post member 164 and thereby increase the size of gap G, which can have the effect of radially compressing the frame 104. To avoid over-crimping the prosthetic heart valve, a stopper 185 (for example, a nut) may be installed on the actuator rod 172 to limit the travel of the actuator rod 172 while rotating the actuator rod 172 to radially compress the frame 104.

In an alternative implementation, as further described herein, some of the actuator rods 172 can be rotated in one direction while the other actuator rods 172 are rotated in an opposite direction simultaneously to either radially expand the frame or radially compress the frame. This counter-rotation of the actuator rods can be used to help reduce the likelihood of the entire frame 104 rotating about the longitudinal axis L during rotation of the actuator rods 172 about their respective axes (for example, when radially expanding the frame 104).

Additional examples of mechanically expandable valves can be found in International Application No. PCT/US2021/052745 and U.S. Provisional Application No. 63/209,904, which are incorporated by reference herein.

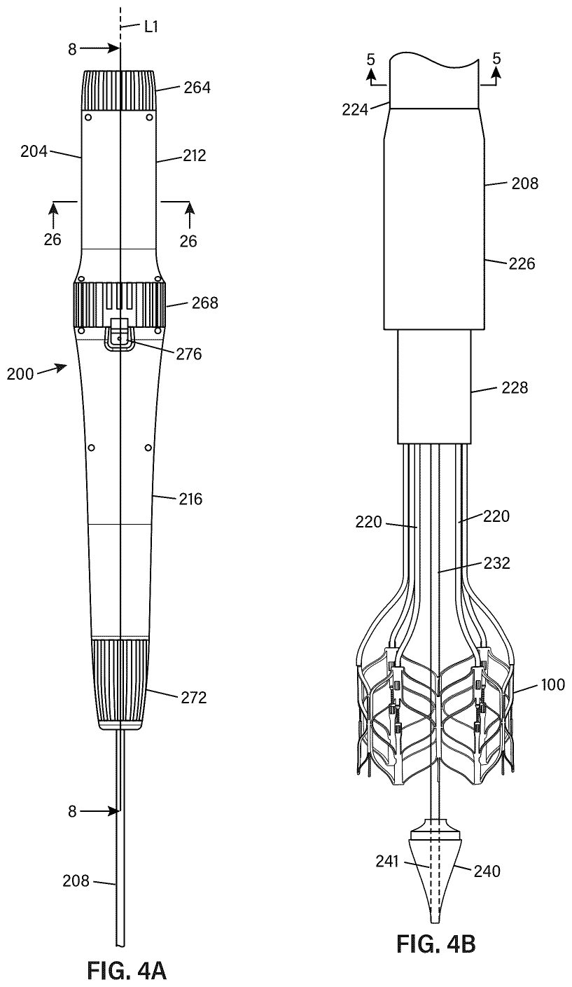

FIGS. 4A and 4B illustrate a delivery apparatus 200, according to some examples. The delivery apparatus 200 can be used to deliver the prosthetic heart valve 100 to an implantation location within a patient's body. The delivery apparatus 200 includes a handle 204 and a shaft assembly 208 coupled to the handle 204. The delivery apparatus 200 can further include one or more actuation assemblies 220 that can be used to releasably couple the prosthetic heart valve 100 to a distal end portion of the shaft assembly 208 and to radially expand and/or compress the prosthetic heart valve 100.

The prosthetic heart valve 100 is shown in an expanded configuration in FIG. 4B. To facilitate delivery of the prosthetic heart valve 100 to an implantation location, the delivery apparatus 200 (and/or other crimping devices) can be used to move the prosthetic heart valve 100 from a radially expanded, functional configuration to a radially compressed, delivery configuration. Once at the implantation location, actuation drivers of the actuation assemblies 220 can operate the actuators 168 of the prosthetic heart valve 100 to radially expand the prosthetic heart valve 100 to a working diameter.

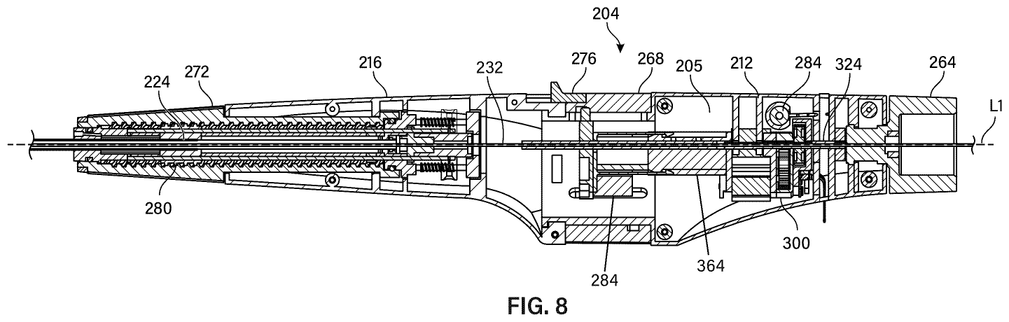

In some examples, the handle 204 includes a proximal body portion 212 and a distal body portion 216 coupled together. The body portions 212, 216 define a cavity (depicted as 205 in FIG. 8) extending along a longitudinal axis L1 of the handle 204. Various mechanisms of the delivery apparatus 200 are disposed within the cavity 205.

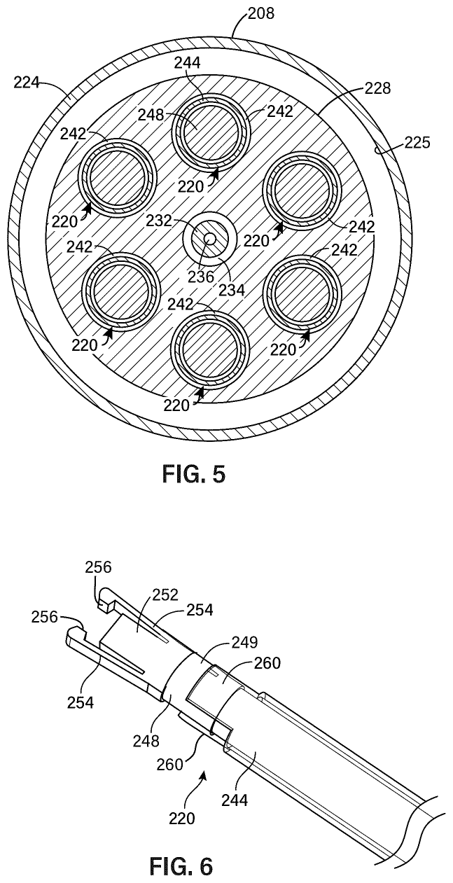

As illustrated in FIGS. 4B and 5, the shaft assembly 208 can include an outer delivery shaft 224 having a lumen 225 extending along the entire length of the shaft. The shaft assembly 208 can include a multi-lumen delivery shaft 228 extending through the lumen 225 and having lumens 234, 242. The shaft assembly 208 can include a nosecone shaft 232 extending through the lumen 234. The actuation assemblies 220 can extend through the lumens 242. The lumen 234 can be centrally disposed within the multi-lumen delivery shaft 228, and the lumens 242 can be angularly spaced apart (uniformly or non-uniformly) about a central axis of the multi-lumen delivery shaft 228 and disposed around the lumen 234.

In some examples, the proximal end portion of the nosecone shaft 232 extends into the portion of the cavity of the handle 204 defined in the proximal body portion 212 (indicated in FIG. 4A), and the distal end portion of the nosecone shaft 232 extends distally from the distal end of the multi-lumen delivery shaft 228 (as shown in FIG. 4B). The prosthetic heart valve 100 can be disposed around the distal end portion of the nosecone shaft 232 when releasably coupled to the actuation assemblies 220.

The nosecone shaft 232 can define a guidewire lumen 236 for receiving a guidewire. As shown in FIG. 4B, a nosecone 240 can be attached to a distal end of the nosecone shaft 232. The nosecone 240 can have a central opening 241 that is aligned and connected to the guidewire lumen 236. During an implantation procedure, a guidewire can be initially inserted into a patient's vasculature. The proximal end of the guidewire can be inserted into the central opening 241 of the nosecone 240 to allow the delivery apparatus 200 to be advanced through the patient's vasculature to an implantation location over the guidewire.

FIG. 6 illustrates a distal end portion of the actuation assembly 220. Each actuation assembly 220 can include an outer sleeve 244 and an actuator driver 248 extending through the outer sleeve 244. In the example, the actuator driver 248 includes a distal head having a central protrusion 252 and one or more flexible elongated elements 254. The central protrusion 252 can be configured to extend into the slot 188 (shown in FIG. 3) of the actuator head 176 of an actuator 168 of the prosthetic heart valve. The flexible elongated elements 254 can have radial protrusions 256 configured to engage the shoulders 192 (shown in FIG. 3) of the actuator head 176.

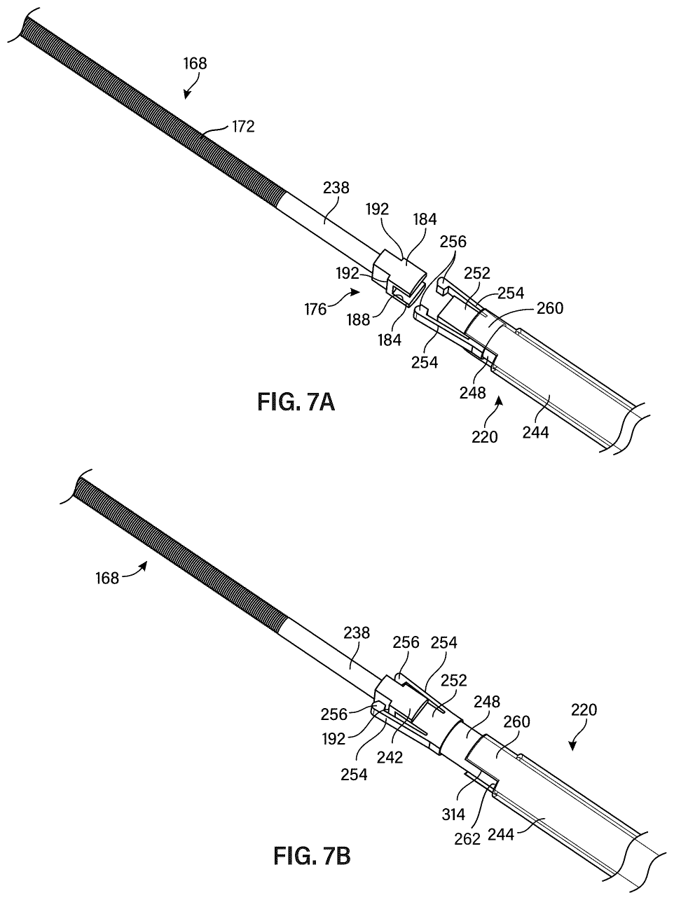

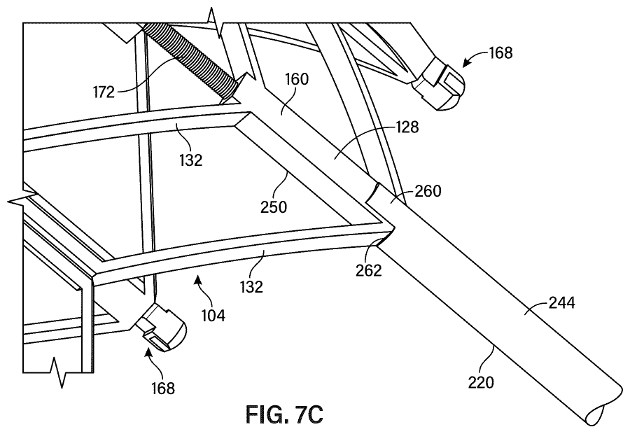

FIGS. 7A-7C illustrate engagement of an actuation assembly 220 with a respective actuator 168. Initially, the distal end portion of the actuation assembly 220 is aligned with the actuator head 176 of the actuator 168, as shown in FIG. 7A. The distal end portion of the actuator driver 248 is then advanced such that the central protrusion 252 of the actuator driver 248 is disposed within the slot 188 of the actuator head 176 of the actuator 168. When the central protrusion 252 is engaged with the slot 188, the flexible elongated elements 254 are disposed at the sides of the actuator head 176, and the radial protrusions 256 of the flexible elongated elements 254 are positioned distally to the shoulders 192 on the actuator head 176, as shown in FIG. 7B.

The outer sleeve 244 can be advanced over the distal end portion of the actuator driver 248 to radially compress the flexible elongated elements 254 against the actuator head 176 until the radial protrusions 256 abut the shoulders 192, thereby coupling the actuator driver 248 to the actuator 168. The outer sleeve 244 can be further advanced until the outer sleeve 244 engages the frame 104, as illustrated in FIG. 7C.

The outer sleeve 244 can have first and second support extensions 260 defining gaps or notches 262 between the extensions 260. As illustrated in FIG. 7C, the support extensions 260 can be oriented such that when the actuation assembly 220 is coupled to a respective actuator 168, the support extensions 260 extend partially over a proximal end portion of the upper post member 160 of the respective support post 128. The engagement of the support extensions 260 with the frame 104 can counteract rotational forces applied to the frame 104 by the actuator rods 172 during expansion of the frame 104.

Various other coupling mechanisms can be used to releasably couple the prosthetic heart valve to the actuation assembly of the delivery apparatus. For example, additional coupling mechanisms are described in International Patent Application PCT/US2022/031257 and U.S. Patent Application No. 63/319,702, which are incorporated by reference herein.

As shown in FIG. 4A, the handle 204 can include one or more knobs that can be configured to perform various functions of the delivery apparatus 200 to deliver the prosthetic heart valve 100 to an implantation location within a patient's body. In some examples, the handle 204 can include a first knob 264, a second knob 268, and a third knob 272. In some examples, the knobs 264, 268, 272 can be knobs that are rotatable about the longitudinal axis L1 of the handle 204 and relative to the body portions 212, 216 of the handle. The handle 204 can include other knobs that can be rotatable or slidable, such as a safety knob 276.

In the example, the first knob 264 is located at a proximal end of the handle 204 and can be used to operate the actuation assemblies 220 of the delivery apparatus 200 and the actuators 168 of the prosthetic heart valve 100. As illustrated in FIG. 8, the first knob 264 can be configured to operate a gearbox 300 disposed within a proximal portion of the cavity 205 of the handle 204. The actuator drivers 248 of the actuation assemblies 220 can be coupled to the gearbox 300 in order to be rotated by the gearbox 300. The rotation of the actuator drivers 248 can be translated to rotational motion of the actuators 168 of the prosthetic heart valve 100.

In the example, the second knob 268 is located where the proximal and distal body portions 212, 216 of the handle 204 are coupled together. The second knob 268 can be configured to release the actuation assemblies 220 from the prosthetic heart valve 100 (for example, after positioning the prosthetic heart valve 100 at the desired implantation location and expanding the prosthetic heart valve 100 to the working diameter). In some examples, the safety knob 276 can be configured to prevent unintentional release of the actuation assemblies 220 from the prosthetic heart valve. For example, the safety knob 276 can slide into a recess in the second knob 268 to prevent rotation of the second knob 268. Retraction of the safety knob 276 from the recess can allow the second knob 268 to be rotated.

In the example, the third knob 272 is located at a distal end of the handle 204. The third knob 272 can be configured such that rotation of the knob relative to the handle body results in the outer delivery shaft 224 moving axially relative to the actuation assemblies 220, the prosthetic heart valve 100, and the nosecone shaft 232.

In some examples, a delivery capsule 226 (shown in FIG. 4B) can be attached to a distal end of the outer delivery shaft 224. Axial movement of the outer delivery shaft 224 in a distal direction relative to the other shafts and prosthetic valve can move the delivery capsule 226 over the distal end portions of the actuation assemblies 220 and the prosthetic heart valve 100 (that is, when the prosthetic heart valve 100 is in the radially compressed configuration) such that the prosthetic heart valve 100 is enclosed within the delivery capsule. Axial movement of the outer delivery shaft 224 in a proximal direction relative to the other shafts and the prosthetic valve can retract the delivery capsule 226 from the prosthetic heart valve 100, exposing the prosthetic heart valve, for example, for deployment at an implantation location. In some examples, the third knob 272 can be operatively coupled to a carriage 280 within the distal portion of the cavity 205 of the handle 204. The outer delivery shaft 224 can be coupled to the carriage 280 such that movement of the carriage 280 due to rotation of the third knob 272 results in axial displacement of the outer delivery shaft 224.

During expansion of the prosthetic heart valve 100, rotation of the actuators 168 can apply moment forces to the frame 104, that is, due to the frictional forces acting between the frame 104 and the actuator rods 172 of the actuators 168. These moment forces can, in some instances, result in the frame 104 rotating or pivoting about the longitudinal axial L of the frame during the expansion/contraction procedure. To help reduce such rotation of the entire frame, the actuators 168 can be divided into two sets, and the two sets can be rotated in opposite directions such that the moment forces due to one set of actuators is counterbalanced by the moment forces due to the other set of actuators. This can, for example, help the frame 104 to remain rotationally fixed or at least substantially rotationally fixed during expansion of the prosthetic heart valve. Thus, this configuration can, for example, make positioning and/or deploying a prosthetic heart valve relatively easier and/or predictable.

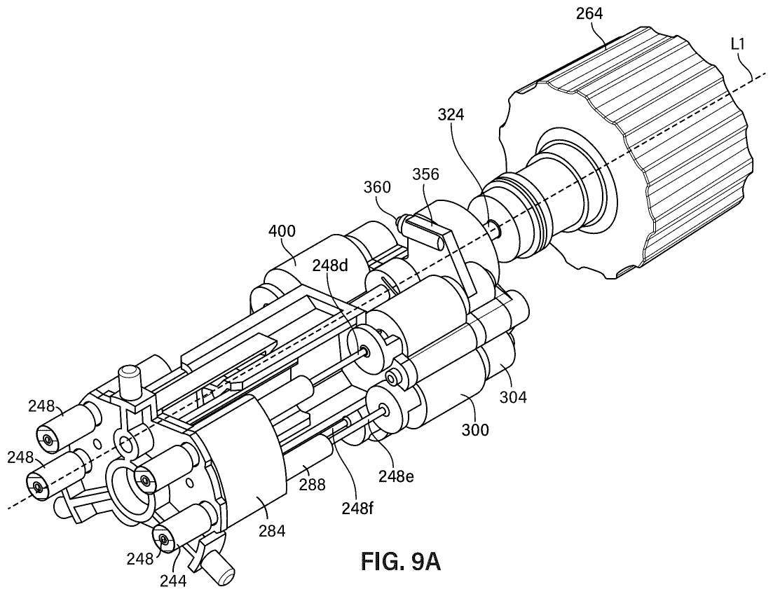

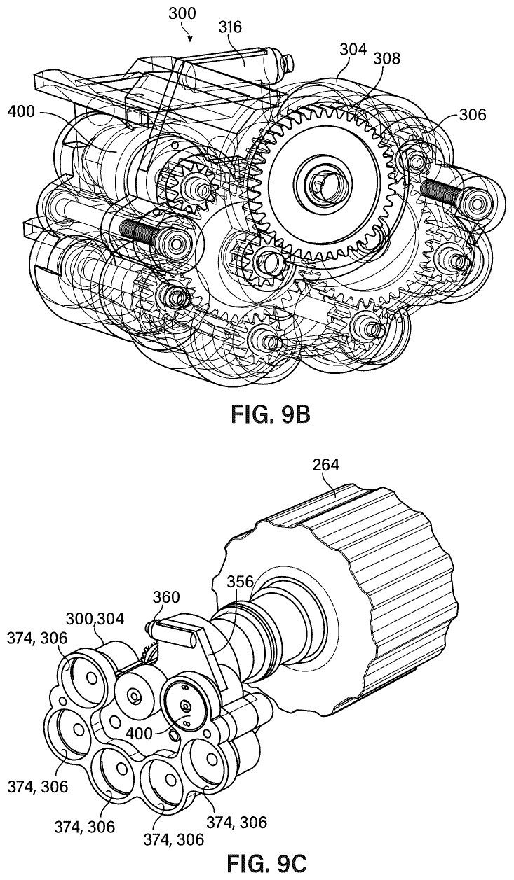

As illustrated in FIGS. 9A-9C, the gearbox 300 of the handle 204 can include a gearbox housing 304 with various compartments 306 to hold the components of a gear train 308. The output shafts of the gear train 308 can be coupled to the actuator drivers 248 such that operation of the gear train 308 results in rotation of the actuator drivers 248 and consequently rotation of the actuators 168 of the prosthetic heart valve 100. In some examples, the gearbox 300 can be a counter-rotation gearbox where the gear train 308 is configured to rotate two sets of actuator drivers in opposite directions.

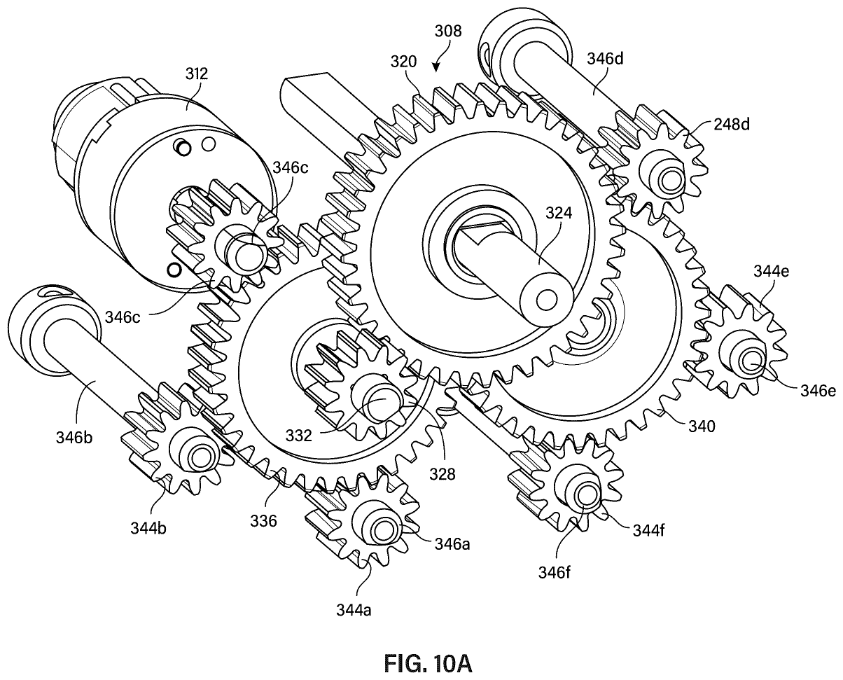

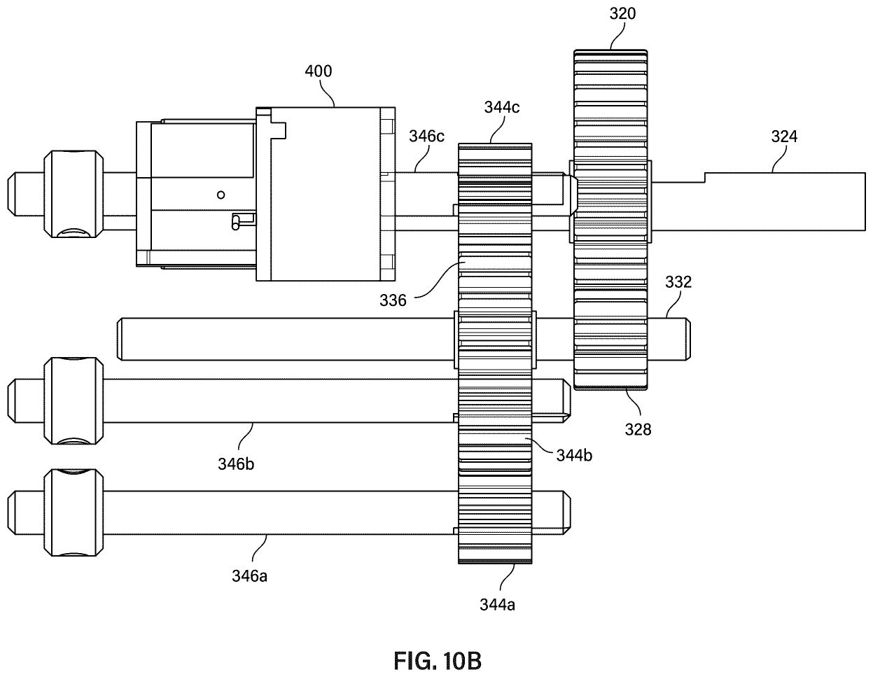

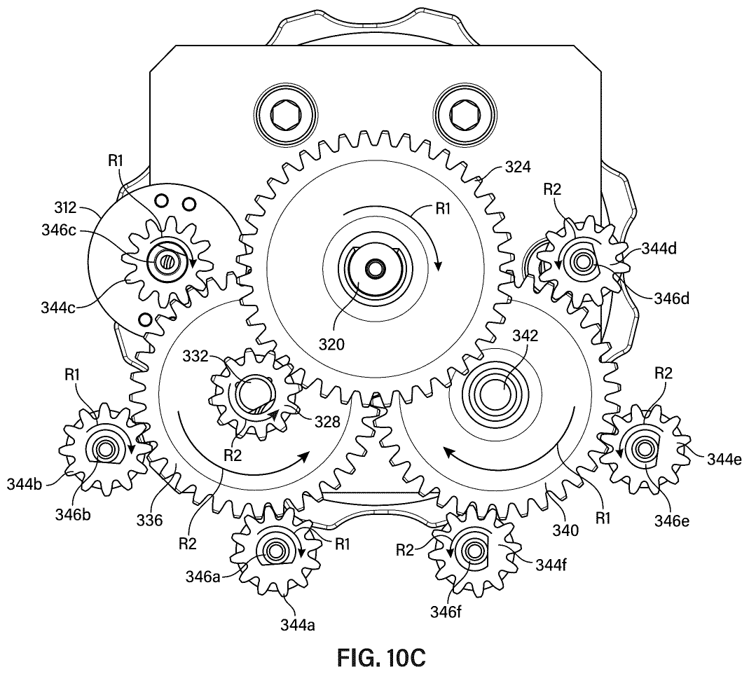

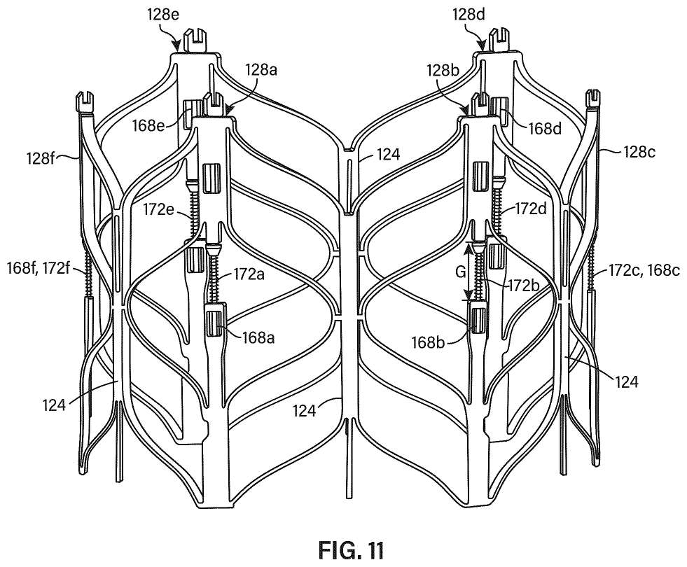

FIGS. 10A-10C illustrate one implementation of the gear train 308. In the example, the gear train 308 includes an input shaft 324 and an input gear 320 coupled to the input shaft 324. In some examples, the input shaft 324 is aligned with the longitudinal axis L1 of the handle 204 (as depicted in FIG. 8). The input shaft 324 can be coupled to the first knob 264 of the handle 204 (as depicted in FIG. 8) such that rotation of the first knob 264 results in rotation of the input shaft 324. The input gear 320 rotates with the input shaft 324. The rotational direction R1 of the input gear 320 can be clockwise or counterclockwise, depending on the direction in which the first knob 264 is rotated.

The gear train 308 can include a transmission gear 328 coupled to a transmission shaft 332, which can be arranged in parallel to the input shaft 324. The teeth of the input gear 320 are meshed with the teeth of the transmission gear 328 such that rotation of the input gear 320 drives the transmission gear 328. The transmission shaft 332 rotates with the transmission gear 328. In some examples, rotation of the input gear 320 in a first direction R1 drives the transmission gear 328 in a second direction R2 that is opposite to the first direction (whether R2 is clockwise or counterclockwise will depend on the rotational direction R1 as determined by the rotation of the first knob 264).

The gear train 308 can include a first driving gear 336 coupled to the transmission shaft 332 and disposed distally to the transmission gear 328. In this case, rotation of the transmission shaft 332 in response to driving the transmission gear 328 by the input gear 320 is translated to rotation of the first driving gear 336. The first driving gear 336 rotates in the same direction R2 as the transmission gear 328.

The gear train 308 can include a second driving gear 340 supported on a driving shaft 342 that is arranged in parallel to the transmission shaft 332. The teeth of the second driving gear 340 are meshed with the teeth of the first driving gear 336 such that rotation of the first driving gear 336 drives the second driving gear 340. The driving shaft 342 rotates with the second driving gear 340. The second driving gear 340 rotates in a direction R1 that is opposite to the direction R2 in which the first driving gear 336 rotates.

The gear train 308 can include a set of first output gears (which can also be referred to as “pinion gears”) angularly spaced apart about a central axis of the first driving gear 336 and having teeth meshed with the teeth of the first driving gear 336. In the example, the set of first output gears includes output gears 344a, 344b, 344c. The output gears 344a, 344b, 344c rotate in a direction R1 that is opposite to the direction R2 in which the first driving gear 336 is rotating. In some examples, the output gears 344a, 344b, 334c are coupled to output shafts 346a, 346b, 346c, respectively. The output shafts 346a, 346b, 346c can be coupled to a first set of actuator drivers.

The gear train 308 can include a second set of output gears (which can also be referred to as “pinion gears”) angularly spaced apart about a central axis of the second driving gear 340 and having teeth meshed with the teeth of the second driving gear 340. In the example, the second set of output gears includes output gears 344d, 344c, 344f. The output gears 344d, 344c, 344f rotate in a direction R2 that is opposite to the direction R3 in which the second driving gear 340 is rotating. As such, the output gears 344d, 344c, 344f of the second set of output gears rotate in a direction that is opposite to the direction in which the output gears 344a, 344b, 344c of the first set of output gears rotate. In some examples, the output gears 344d, 344c, 344f are coupled to output shafts 346d, 346e, 346f, respectively. The output shafts 346d, 346e, 346f can be coupled to a second set of actuator drivers.

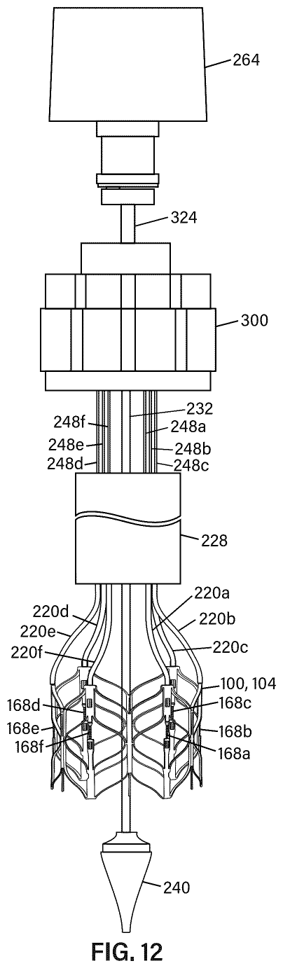

For illustrative purposes, FIG. 11 shows the frame 104 with actuators 168a, 168b, 168c, 168d, 168e, 168f coupled to support posts 128a, 128b, 128c, 128d, 128c, 128f, respectively. In some examples, a first set of actuators can include actuators 168a, 168b, 168c, and a second set of actuators can include actuators 168d, 168e, 168f. The first set of actuators 168a, 168b, 168c can be coupled to the first set of actuator drivers 248a, 248b, 248c, and the second set of actuators 168d, 168e, 168f can be coupled to the second set of actuator drivers 248d, 248e, 248f, as illustrated in FIG. 12 (several details of the delivery apparatus are not shown in FIG. 12 for simplicity; for example, the body of the handle 204 and the outer delivery shaft 224 through which the multi-lumen delivery shaft 228 extends are not shown).

Returning to FIG. 11, the actuator rods 172a, 172b, 172c of the actuators 168a, 168b, 168c in the first set of actuators can have threads with a first configuration (for example, right-hand threads). The actuator rods 172d, 172e, 172f of the actuators 168d, 168e, 168f in the second set of actuators can have threads with a second configuration (for example, left-hand threads) that are opposite to the first configuration. For example, if the actuator rods 172a, 172b, 172c have right-hand threads, the actuator rods 172d, 172e, 172f can have left-hand threads (or vice versa). Thus, when the actuators 168a, 168b, 168c in the first set and the actuators 168d, 168e, 168f in the second set are simultaneously rotated in opposite directions, all the actuators will act in concert to either increase the respective gap G or decrease the gap G.

Other examples of dividing the actuators into two sets are possible. For example, a first set of actuators could include actuators 168a, 168c, 168e, and a second set of actuators could include actuators 168b, 168d, 168f (that is, alternating actuators around the circumference of the frame could be included in a set). In this case, the actuator rods 172a, 172c, 172e of the first set of actuators can have threads with a first configuration (for example, right-hand threads), and the actuator rods 172b, 172d, 172f of the second set of actuators can have threads with a second configuration that is opposite to the first configuration (for example, left-hand threads).

Examples have been given with the prosthetic heart valve 100 having six actuators divided into two sets. In other examples, the prosthetic heart valve could have greater than six (for example, 7-15) or fewer than six (for example, 1-5) actuators. In other cases, the prosthetic heart valve could have an odd number of actuators, in which case one set of actuators could have a greater number of actuators compared to the other set of actuators. The number of actuation assemblies/actuator drivers of the delivery apparatus can generally match the number of actuators of the prosthetic heart valve.

In the simplified illustration of FIG. 12, each of the actuator drivers 248a, 248b, 248c in the first set of actuator drivers extends through the multi-lumen delivery shaft 228 and is connected to a respective actuator 168a, 168b, 168c of the prosthetic heart valve 100. Similarly, each of the actuator drivers 248d, 248e, 248f in the second set of actuator drivers extends through the multi-lumen delivery shaft 228 and is connected to a respective actuator 168d, 168e, 168f of the prosthetic heart valve 100. The actuator drivers 248a, 248b, 248c are coupled to the first set of output shafts of the gearbox 300 (346a, 346b, 346c in FIGS. 10A-10C), and the actuator drivers 248d, 248e, 248f are coupled to the second set of output shafts of the gearbox 300 (346d, 346e, 346f in FIGS. 10A-10C). The input shaft 324 of the gearbox 300 is coupled to the first knob 264.

To radially expand the prosthetic heart valve 100, for example, at an implantation location, the first knob 264 can be used to rotate the first set of actuator drivers 248a, 248b, 248c and the second set of actuator drivers 248d, 248e, 248f in opposite directions. The counter-rotation of the two sets of actuator drivers results in counter-rotation of the first set of actuators 168a, 168b, 168c and the second set of actuators 168d, 168e, 168f. This counter-rotation of the two sets of actuators can advantageously help reduce the likelihood of the prosthetic heart valve rotating relative to the native anatomy during expansion of the prosthetic heart valve.

In some implementations, a torque limit can be defined for each actuator driver 248, and one or more torque limiters can be provided (for example, one for each actuator driver 248) to prevent torque on the actuator driver 248 from exceeding the predefined limit. The torque limiter can, for example, prevent overloading of the actuator driver 248 during expansion of the prosthetic heart valve 100. In some examples, the torque limiter restricts rotation of the corresponding actuator driver 248 when the torque on the actuator driver 248 has reached a predefined limit. Since all the actuator drivers 248 are coupled to the gear train 308, the gear train 308 effectively halts when any of the actuator drivers 248 is stopped by the torque limiter.

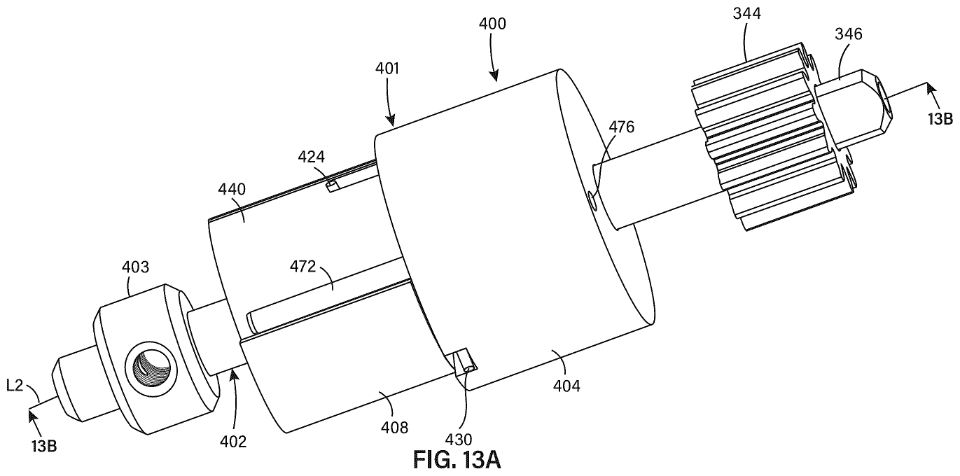

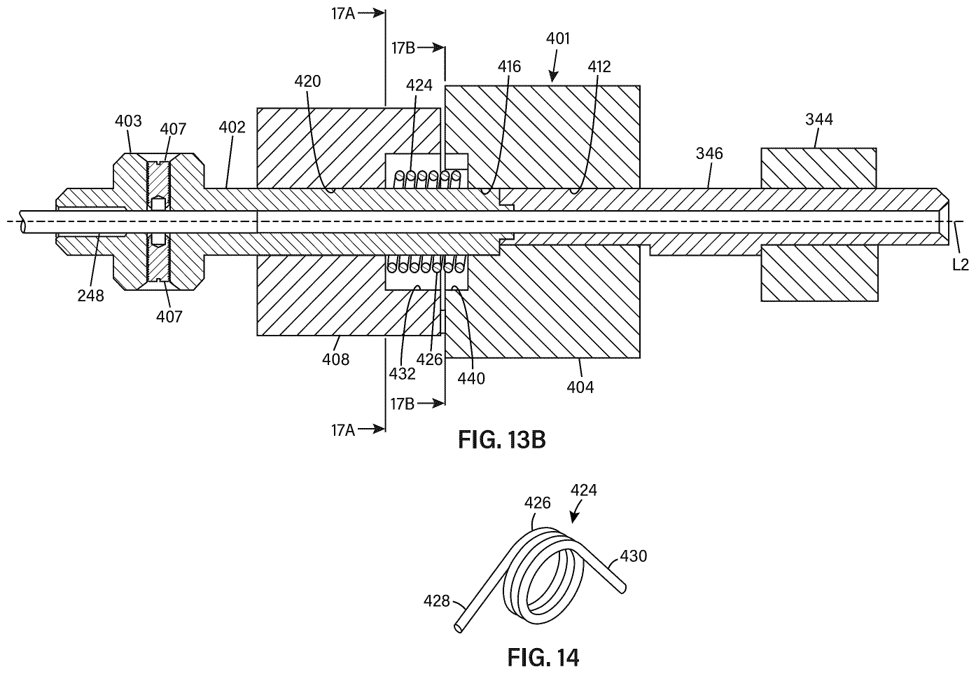

FIGS. 13A and 13B illustrate a torque limiter 400, according to some examples. The torque limiter 400 can couple an actuator driver 248 to an output shaft 346 of the gear train 308 and can operate to prevent rotation of the actuator driver 248 when a torque on the actuator driver 248 is within a predetermined torque limit range. The upper limit of the predefined torque limit range can be a maximum torque on the actuator driver 248, and the lower limit of the predetermined torque limit range can be a torque that is within a tolerance of the maximum torque (for example, within 15% of the maximum torque). In some examples, the maximum torque on the actuator driver 248 can be 50 N-mm. The torque limiter 400 can be housed within a compartment of the gearbox housing 304. For illustrative purposes, FIG. 9B shows the torque limiter 400 within one of the compartments 306 of the gearbox housing 304. Although only one torque limiter 400 is depicted in FIGS. 9B and 9C, in some examples, the handle 204 for the delivery apparatus 200 can comprise a plurality (for example, 2-15) of torque limiters 400. For example, each actuation driver 248 of the delivery apparatus could have a respective torque limiter 400.

Returning to FIGS. 13A-13B, the torque limiter 400 has a longitudinal axis L2. The torque limiter 400 includes a rotatable assembly 401 aligned with and rotatable about the longitudinal axis L2. The rotatable assembly 401 couples a connector shaft 402 to one of the output shafts 346 of the gearbox 300. An output gear 344 is coupled to the output shaft 346, as previously described. One of the actuator drivers 248 can be coupled to the connector shaft 402 at a coupling section 403 of the connector shaft 402 (for example, using one or more set screws 407). In one mode, the rotatable assembly 401 allows the connector shaft 402 to rotate with the output shaft 346. In another mode, the rotatably assembly 401 prevents rotation of both the connector shaft 402 and the output shaft 346.

The rotatable assembly 401 includes a first rotatable body 404 and a second rotatable body 408. In the example, the second rotatable body 408 is positioned distally to the first rotatable body 408, and both the first and second rotatable bodies 404, 408 are rotatable about the longitudinal axis L2. The first rotatable body 404 is fixedly coupled to the output shaft 346 such that the first rotatable body 404 and the output shaft 346 can rotate together about the longitudinal axis L2. In the example, the first rotatable body 404 is positioned distally to the output gear 344. The second rotatable body 408 is fixedly coupled to the connector shaft 402 such that the second rotatable body 408 and the connector shaft 402 can rotate together about the longitudinal axis L2.

In some examples, the first rotatable body 404 includes a proximal axial bore 412 and a distal axial bore 416. A distal end portion of the output shaft 346 is inserted into the proximal axial bore 412 and engages the proximal axial bore 412 in a manner that allows the first rotatable body 404 to rotate with the output shaft 346. In some examples, the proximal axial bore 412 can have a non-circular cross-sectional profile (taken in a plane perpendicular to the longitudinal axis L2) that is adapted to match with a non-circular cross-sectional profile (taken in a plane perpendicular to the longitudinal axis L2) on the input shaft 346 such that rotation of the input shaft 346 results in rotation of the first rotatable body 404. For example, the non-circular cross-sectional profile of the proximal axial bore 412 can be “D shaped” (which can also be referred to as having a “flat”) that can engage a similarly D-shaped (or “flat”) output shaft 346 and allow the first rotatable body 404 to rotate in the same direction as the output shaft 346. Alternatively, the output shaft 346 can be attached to the proximal axial bore 412 (for example, by other means for fixedly coupling such as welding, gluing, and the like) to allow the first rotatable body 404 to rotate with the output shaft 346.

The second rotatable body 408 can include an axial bore 420 that is aligned with the distal axial bore 416 of the first rotatable body 404. The connector shaft 402 extends through the axial bore 420 of the first rotatable body 404 into the distal axial bore 416 of the first rotatable body 404. The connector shaft 402 can engage the second rotatable body 408 in a manner that allows the connector shaft 402 to rotate with the second rotatable body 408. For example, the axial bore 420 can have a non-circular profile to engage a complementary non-circular profile on the connector shaft member 402. Alternatively, the connector shaft 402 can be attached to the axial bore 420 (for example, by welding, gluing, and the like) to allow the second rotatable body 408 to be rotatable with the connector shaft 402. In some examples, the distal end of the output shaft 346 and the proximal end of the connector shaft 402 can be axially spaced apart (for example, separated by a wall or shoulder of the first rotatable body 404).

In other examples, the opposing ends of the connector shaft 402 and output shaft 346 can axially overlap. In such example, the shafts 402, 346 can include one or more features that facilitate alignment of the connector shaft 402 with the output shaft 346 along the longitudinal axis L2 while also allowing relative rotational movement between the connector shaft 402 and the input shaft 346. For example, in some instances, the connector shaft 402 (or at least a portion thereof) can comprise an outer diameter that is smaller than a diameter of an internal bore of the output shaft 346 such that the connector shaft can extend axially into the output shaft 346 (or vice versa).

In any event, the output shaft 346 and the connector shaft 402 are not fixedly coupled together. Thus, in some instances, which are further explained below, the output shaft 346 (and the first rotatable body 404) and the connector shaft 402 (and the second rotatable body 408) can rotate relative to each other.

In the example, the first rotatable body 404 and the second rotatable body 408 are coupled together by a rotational biasing member (for example, a torsion spring 424). As illustrated in FIG. 14, the torsion spring 424 can be a helical torsion spring including a coil portion 426 terminating at opposite ends in first and second end (or arm) portions 428, 430. The first and second end portions 428, 430 of the torsion spring 424 can extend radially outward beyond the coil portion 426 of the torsion spring 424. The torsion spring 424 can be configured such that the first end portion 428 is rotationally offset from the second end portion 430.

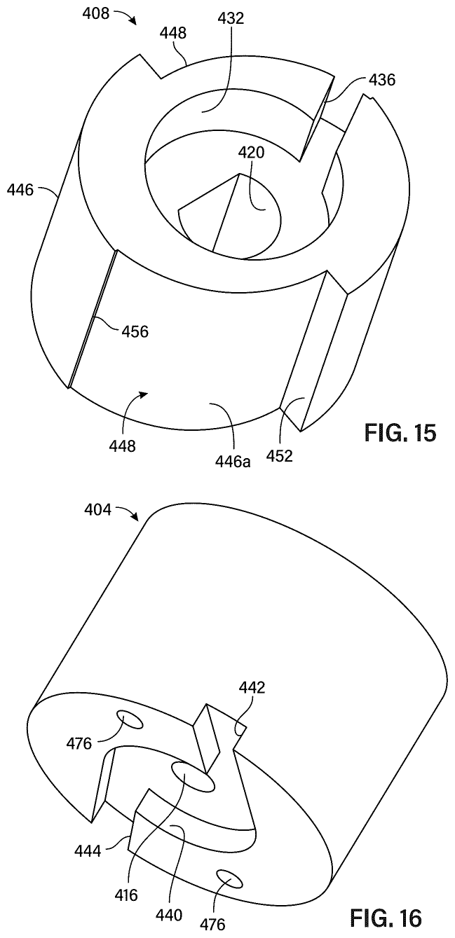

In some examples, as illustrated in FIG. 15, the proximal end portion of the second rotatable body 408 can include a recess 432 and a connected lateral slot 436. The recess 432 can be centrally aligned with the longitudinal axis L2 and connected to the axial bore 420. As illustrated in FIG. 16, the distal end portion of the first rotatable body 404 can include a recess 440 and connected lateral slots 442, 444. The recess 440 can be centrally aligned with the longitudinal axis L2 and connected to the distal axial bore 416. The lateral slots 442, 444 are rotationally offset from each other about the longitudinal axis L2. As shown in FIG. 13B, the connector shaft 402 can extend through the recesses 432, 440 while passing through the axial bore 420 into the distal axial bore 416.

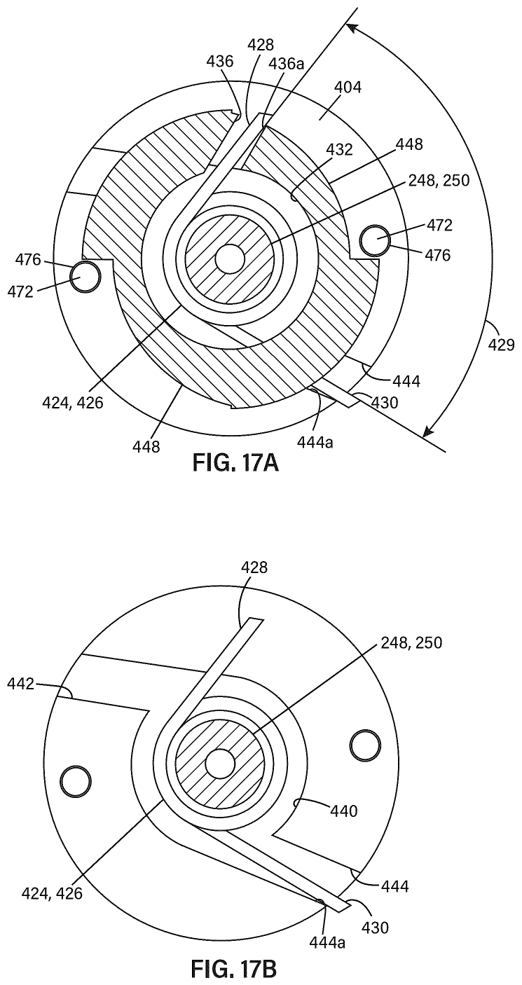

The coil portion 426 of the torsion spring 424 can be arranged in the chamber formed by the aligned recesses 432, 440 with the first end portion 428 extending into the connected lateral slot 436 (as illustrated in FIG. 17A) and the second end portion 430 extending into one of the lateral slots 442, 444 in the first rotatable body 404 (as illustrated in FIG. 17B). In this position, the coil portion 426 is disposed around the portion of the connector shaft 402 extending through the recesses 432, 440 (depicted in FIG. 13B). The central axis of the coil portion 426 is aligned with the longitudinal axis L2 of the torque limiter 400 such that both the rotatable bodies 404, 408 can rotate about the central axis of the coil portion 426.

As illustrated in FIG. 17A, the end portions 430, 428 of the torsion spring 424 can engage surfaces 444a, 436a of the respective receiving slots 444, 436 formed in the rotatable bodies 404, 408. The torsion spring 424 can bias the rotatable bodies 404, 408 into an initial position in which the rotatable bodies 404, 408 rotate together as a single body. The torsion spring 424 is configured to twist in a direction in which the end portions 430, 428 approximate each other when the torque on the actuator driver 248 is within predefined torque limit range. In some cases, the torsion spring 424 can be preloaded, and the torsion spring 424 can start twisting when the torque on the actuator driver 248 exceeds the preload in the torsion spring 424. The preload in the torsion spring 424 can be set as the lower limit of the predetermined range. The upper limit of the predetermined range can be the predetermined torque limit on the actuator driver 248, and the lower limit of the predetermined range can be less than the predetermined torque limit on the actuator driver 248 (for example, within 10 to 15% of the predetermined torque limit). This means that the torsion spring 424 will start twisting as the actuator driver 248 approaches the predetermined torque limit rather than after the actuator driver 248 reaches or exceeds the predetermined torque limit. In some cases, the predetermined torque limit can be 50 N-mm. The initial angular spacing 429 illustrated in FIG. 17A corresponds to the initial position of the rotatable bodies 404, 408. The angular spacing 429 becomes smaller as the end portions 430, 428 approximate each other during twisting of the torsion spring 424.

As illustrated in FIG. 15, tapered recessed portions 448 can be formed on the outer surface 446 of the second rotatable body 408. In the example, each tapered recessed portion 448 includes a first radial shoulder 452, a second radial shoulder 456 spaced from the first radial shoulder 452 in a circumferential direction of the second rotatable body 408, and a portion 446a of the outer surface 446 between the first and second radial shoulders 452, 456. The outer surface portion 446a can be a curved surface in some examples.

The radial projection of the first radial shoulder 452 is greater than the radial projection of the second radial shoulder 456 such that the recessed portion 448 tapers in the radial direction (that is, deep to shallow) from the first radial shoulder 452 to the second radial shoulder 456. Each tapered recessed portion can extend axially along the entire length of the second rotatable body 408 or partially along the length of the second rotatable body 408. In some examples, two tapered recessed portions 448 are formed on the outer surface 446. The tapered recessed portions 448 are angularly spaced from each other about a central axis of the second rotatable body 408, which can be the same as the longitudinal axis L2 of the torque limiter. The angular spacing between the two tapered recessed portions 448 can be such that the two tapered recessed portions are diametrically opposed about the central axis of the second rotatable body 408.

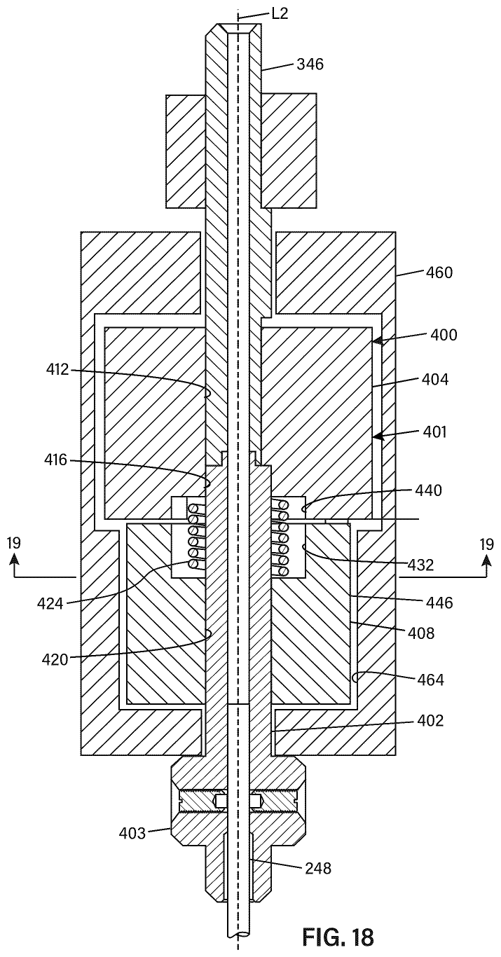

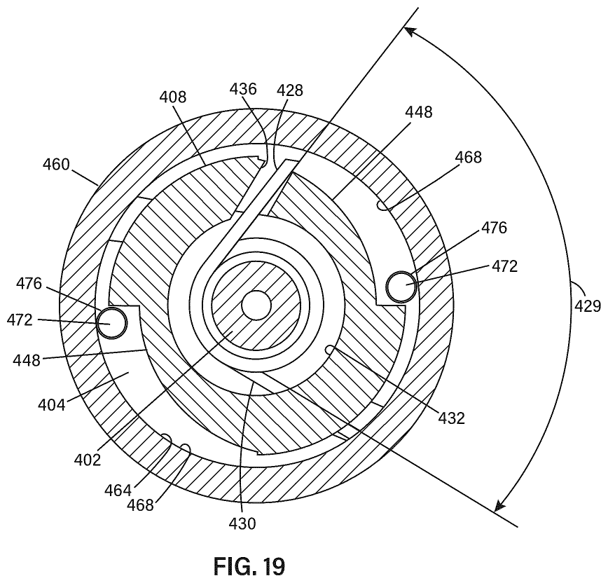

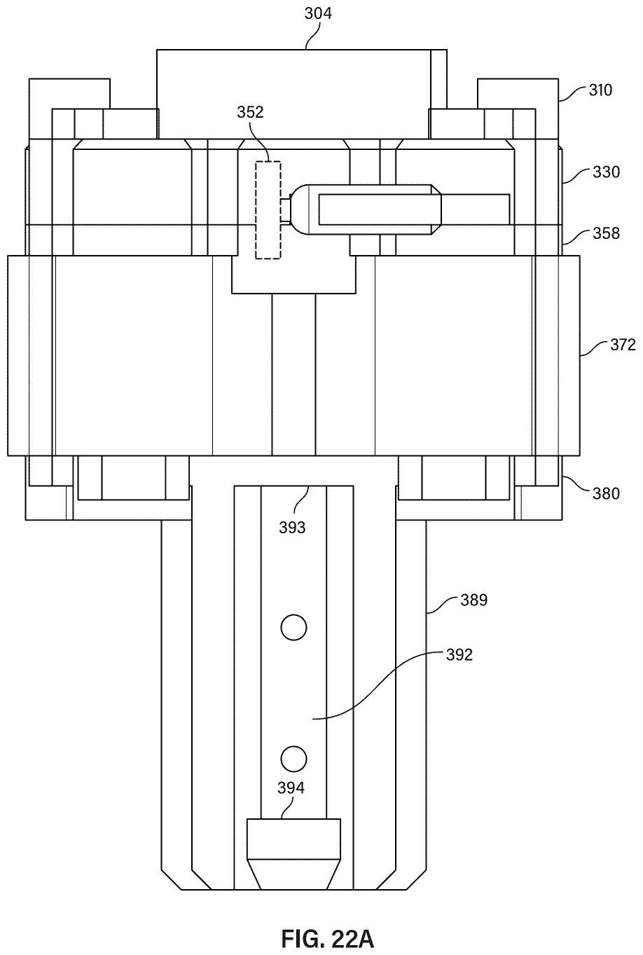

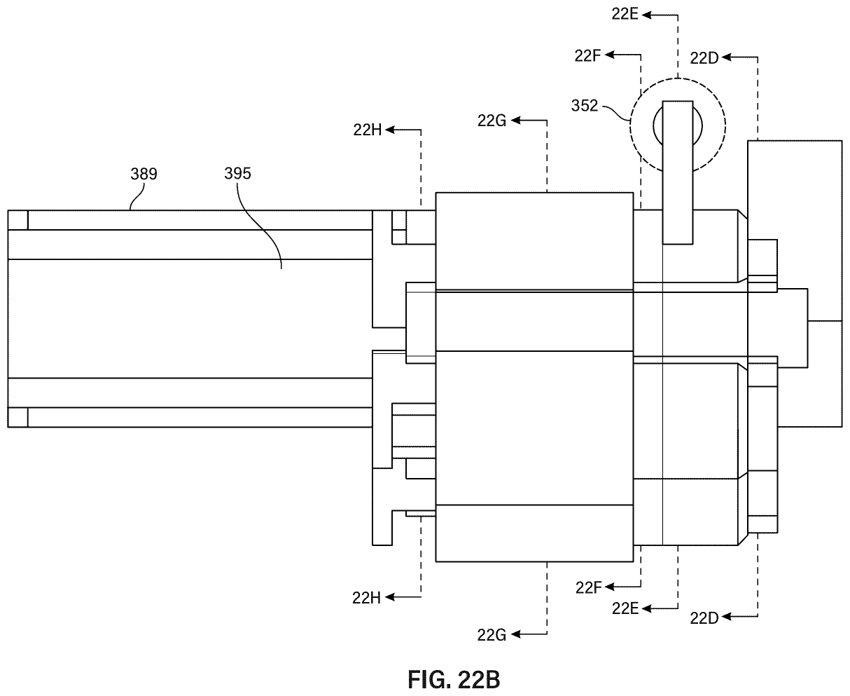

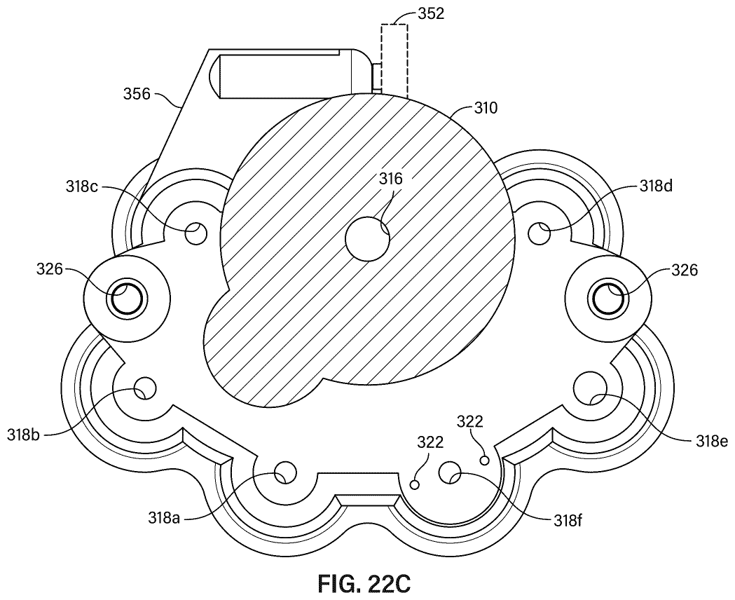

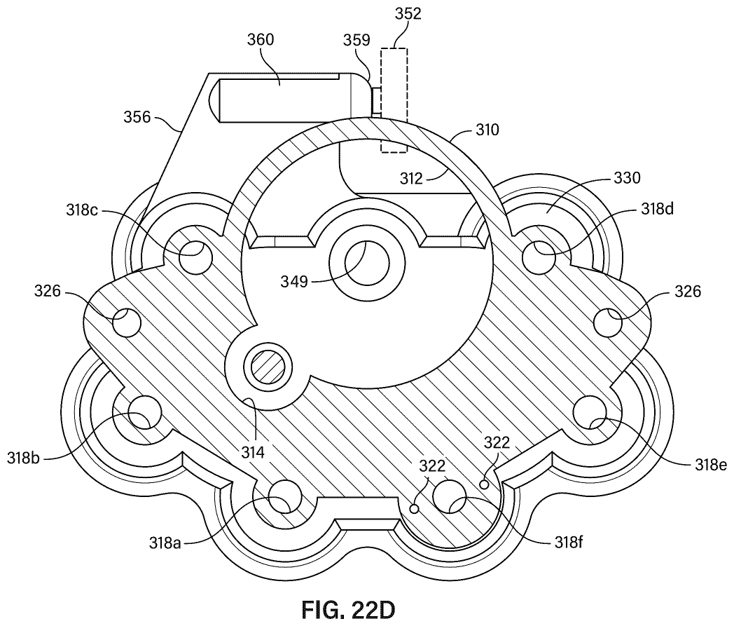

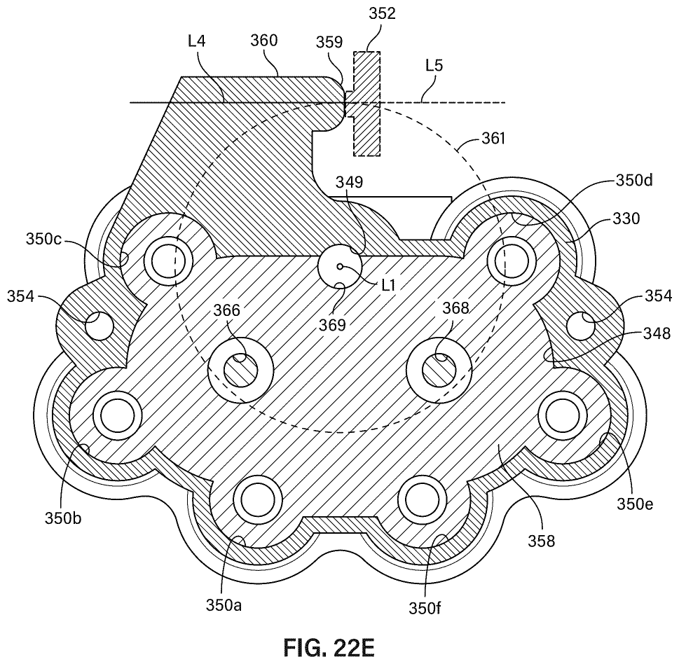

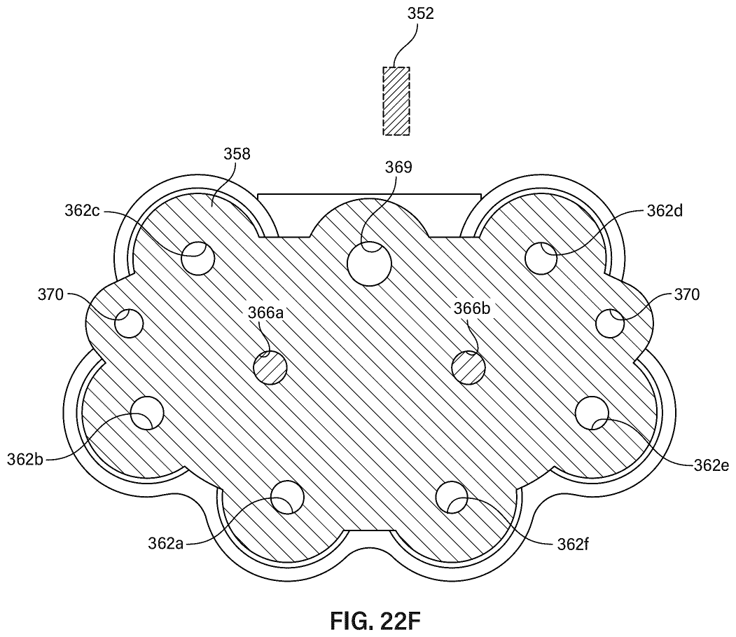

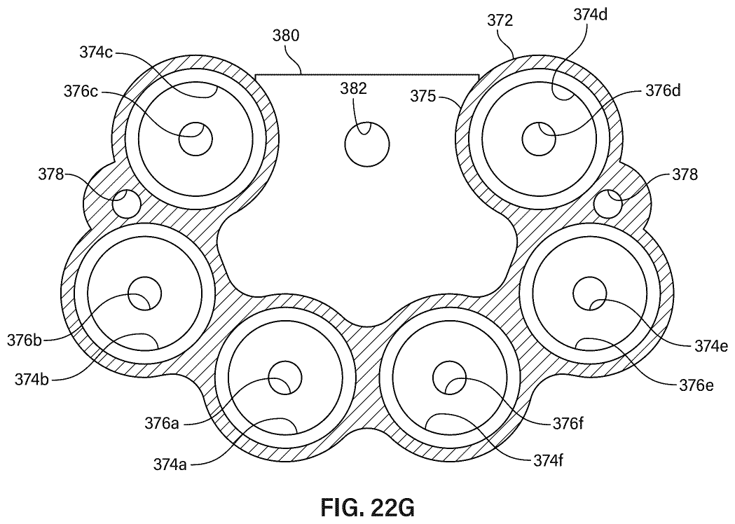

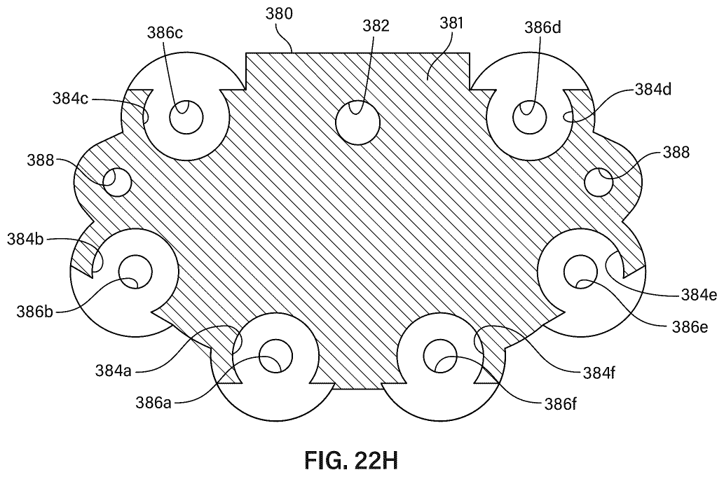

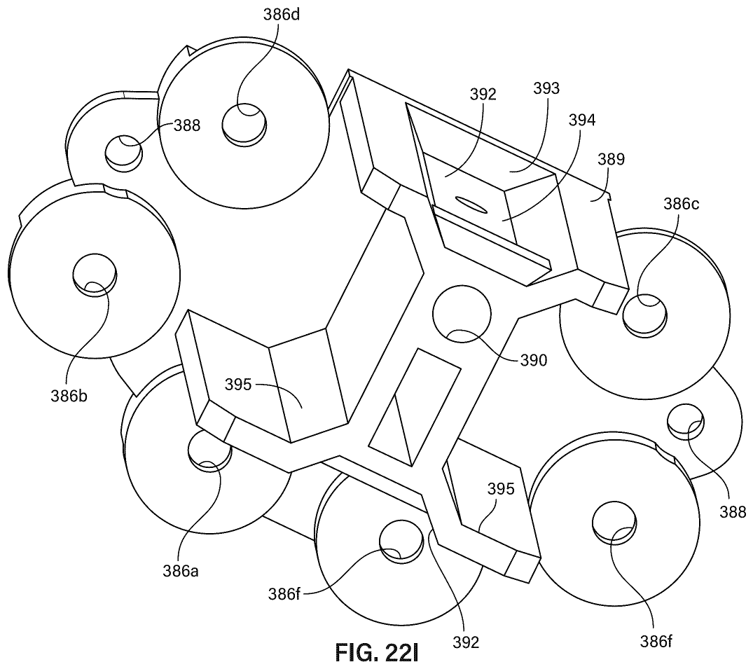

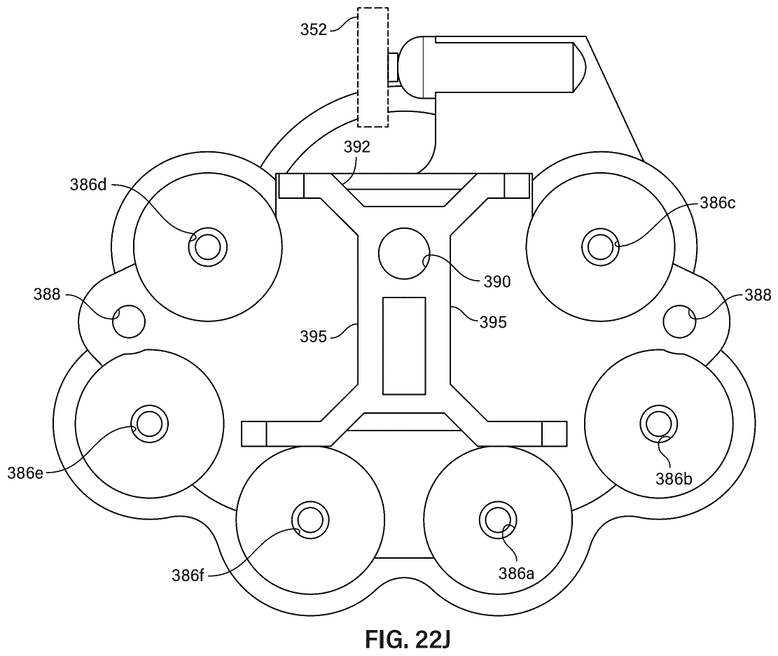

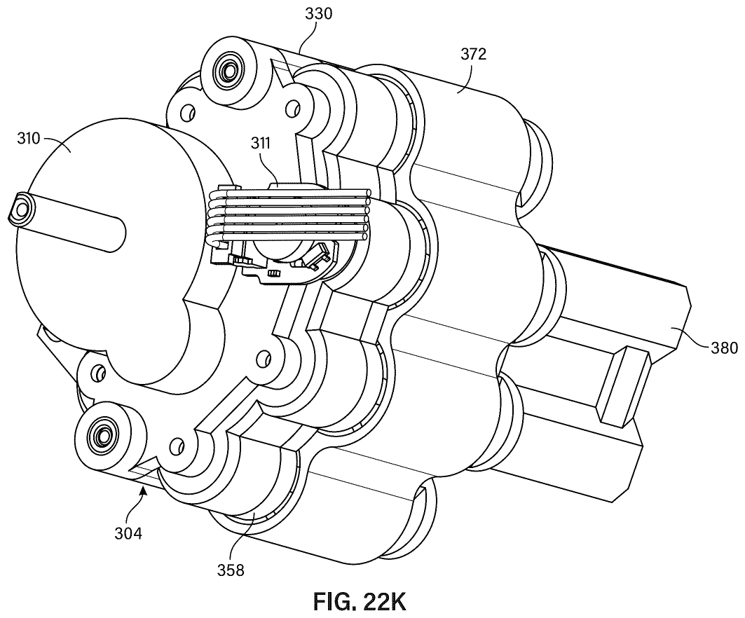

As further illustrated in FIG. 18, the rotatable assembly 401 of the torque limiter 400 can be disposed in a housing 460 such that the outer surface 446 of the second rotatable body 408 is circumscribed by an inner surface 464 of the housing 460. The tapered recessed portions 448 in the outer surface 446 and the inner surface 464 can define circumferentially tapered channels 468 disposed on the periphery of the second rotatable body 408, as illustrated more clearly in FIG. 19. The housing 460 can be a compartment of the gearbox housing 304 (for example, one of compartments 374a-f illustrated in FIG. 22G) or can be a separate housing that is mounted to the gearbox housing 304.

As shown more clearly in FIG. 19, each channel 468 accommodates a wedge member 472. In some examples, the wedge member 472 can be in the form of a longitudinal rod member. In some examples, the wedge members 472 are fixedly coupled to the first rotatable body 404 such that the wedge members 472 rotate with the first rotatable body 404. In some examples, proximal portions of the wedge members 472 extend into longitudinal holes 476 in the first rotatable body 404 (shown in FIGS. 13A, 16, 17A, 17B, 19). The wedge members 472 can be held in place in the holes 476 using any suitable method (for example, by friction, welding, gluing, and the like).

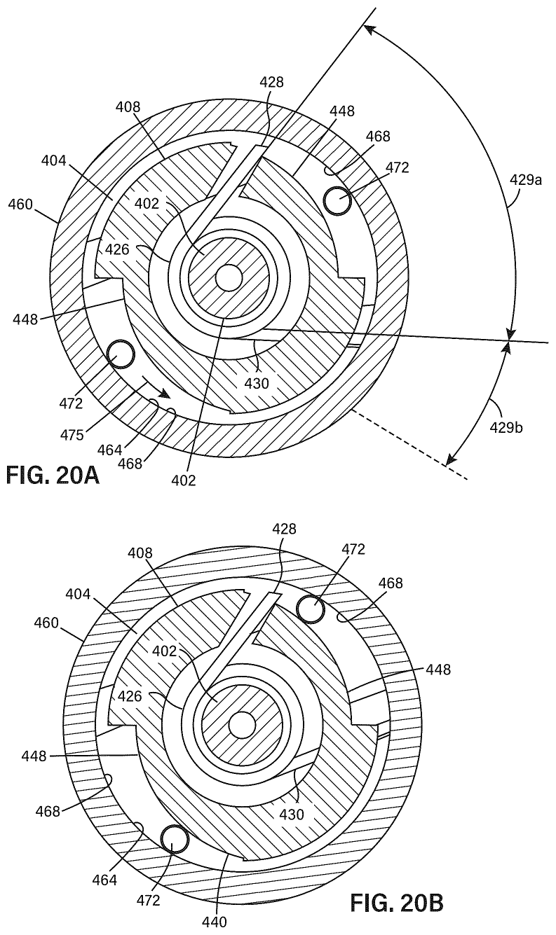

FIGS. 19, 20A, and 20B illustrate operation of the torque limiter 400. The first knob 264 of the handle 204 can be rotated to operate the gear train 308. While the gear train 308 is working, the gear train 308 rotates the output shaft 346. The first rotatable body 404 rotates with the output shaft 346. Rotation of the first rotatable body 404 is translated to rotation of the second rotatable body 408 through the torsion spring 424. As the second rotatable body 408 rotates, the actuator driver 248, which is coupled to the second rotatable body 408 via the connector shaft 402, also rotates. In the state shown in FIG. 19, the torsion spring 424 is in its resting, undeflected state, with an initial angular difference 429 between the end portions 428, 430. In this state, the wedge members 472 are freely accommodated in the wide end of the channels 468, and the first and second rotatable bodies 404, 408 rotate together.

If a torque on the actuator driver 248 reaches a predefined torque limit range set by the size and properties of the torsion spring 424, the coil portion 426 of the torsion spring 424 twists in a manner that approximates the end portions 428, 430 of the torsion spring 424 towards each other. FIG. 20A illustrates an angular spacing 429a between the end portions 428, 430 that is smaller than the initial angular spacing 429 (depicted in FIG. 19) due to the end portions 428, 430 approximating each other (the initial angular spacing 429 shown in FIG. 19 is the sum of the angular spacings 429a, 429b indicated in FIG. 20A). As the torsion spring 424 twists, the first rotatable body 404 rotates relative to the second rotatable body 408 (rather than rotating together), as illustrated in FIG. 20A. As the first rotatable body 404 rotates relative to the second rotatable body 408, the wedge members 472 move along the tapered channels 468 in a direction from the wide end of the channels to the narrow end of the channels, as illustrated by the arrow 475.

The first rotatable body 404 stops rotating when the wedge members 472 are pressed against the narrow end of the channels 468 such that further rotational movement of the wedge members 472 within the tapered channels 468 is not possible due to the interference between the surfaces of the housing 460 and the second rotatable body 408 and the wedge members 472, as illustrated in FIG. 20B. In this state, the second rotatable body 408 also stops rotating. Since all the gears of the gear train 308 are interconnected, once the actuator driver 248 reaches a torque limit that halts rotation of the first and second rotatable bodies 404, 408 and the output gear 344 associated with the actuator driver 248, the movement of the entire gear train 308 stops, preventing rotational movement of all other actuator drivers 248 coupled to the gearbox 300.

In this manner, the torque limiter 400 can help ensure that the actuation members and/or other components of the prosthetic heart valve and/or delivery apparatus are operated within the predetermined torque limits. This can, among other things, reduce or prevent the prosthetic heart valve from being damaged during expansion/contraction and/or prevent the prosthetic heart valve from being overly expanded relative to a native annulus (and/or other native tissue).

The gearbox housing 304 can include various compartments to accommodate the components of the gear train 308 and torque limiter 400, as illustrated in FIGS. 22A-22J.

In some examples, as shown in FIG. 22A-22D, the gearbox housing 304 can have a first housing section 310 forming a proximal end portion of the gearbox housing. The first housing section 310 can include compartments 312 and 314 to accommodate the input gear 320 (shown in FIGS. 10A-10C) and the transmission gear 328 (shown in FIGS. 10A-10C), respectively. The first housing section 310 can include a hole 316 for passage of a proximal end portion of the input shaft 324 (for example, to allow coupling of the proximal end portion of the input shaft 324 to the first knob 264 (shown in FIG. 12)). The first housing section 368 can include holes 318a-f for passage of proximal end portions of the output shafts 346a-f (shown in FIGS. 10A-10C). The first housing section 310 can further include fastening holes 326 (shown in FIG. 22C) that can receive fasteners, such as bolts, which can be used to fasten the first housing section 310 to other housing sections of the gearbox housing.

In some cases, the first housing section 310 can include mounting holes 322 for mounting of an encoder about a proximal end portion of one of the output shafts 346a-f. For example, the mounting holes 322 can receive fasteners, such as screws, that are used to attach the encoder to the first housing section 310 and around the respective output shaft. FIG. 22K shows an encoder 311 mounted on one of the output shafts. In some examples, the encoder 311 can include a sensing member that can detect the number of rotations of the output shaft. In some examples, the encoder can be a magnetic encoder including a magnetic sensor and a magnetic arrangement to generate a magnetic field. The magnetic sensor can detect changes in the magnetic field as the output shaft rotates. Other types of encoders can be used, such as optical encoders.

In some examples, as shown in FIGS. 22A-22E, the gearbox housing 304 can have a second housing section 330 disposed adjacent to the first housing section 310. The second housing section 330 includes a central opening 348 and compartments 350a-f formed on the periphery of the central opening 348. The central opening 348 can accommodate the driving gears 336, 340 (shown in FIGS. 10A-10C). The compartments 350a-f can accommodate the output gears 344a-f (shown in FIGS. 10A-10C). The compartments 350a-f are longitudinally aligned with the holes 318a-f in the first housing section 310. The second housing section 330 can include fastening holes 354 that can be aligned with the fastening holes 326 in the first housing section 310 to receive fasteners. The second housing section 330 can include an opening 349 that is aligned with the compartment 312 in the first housing section 310. The opening 349 can allow the input shaft 324 to extend through the second housing section 330 when the input gear 320 is mounted in the compartment 312.

In some examples, as shown in FIGS. 22A, 22B, 22E, and 22F, the gearbox housing 304 can have a third housing section 358 disposed adjacent to the second housing section 330 and forming an end wall for the central opening 348 and compartments 350a-f in the second housing section 330. The third housing section 358 can include holes 362a-f (shown in FIG. 22F) to receive the output shafts 346a-f (shown in FIGS. 10A-10C) when the output gears 344a-f (shown in FIGS. 10A-10C) are disposed in the compartments 350a-f of the second housing section 330. The third housing section 358 can include holes 366a and 366b to receive the transmission shaft 332 (shown in FIGS. 10A-10C) and the driving shaft 342 when the driving gears 336, 340 are disposed in the central opening 348 of the second housing section 330. The third housing section 358 can include an opening 369 that is aligned with the opening 349 in the second housing section 330. The opening 369 can allow the input shaft 324 to extend through the third housing section 358 when the input gear 320 is mounted in the compartment 312 of the first housing section 310. The third housing section 358 can include fastening holes 370 that can be aligned with the fastening holes 354 in the second housing section 330 and the fastening holes 326 in the first housing section 310 to receive fasteners.

In some examples, as shown in FIGS. 22A, 22B, and 22G, the gearbox housing 304 can have a fourth housing section 372 disposed adjacent to the third housing section 358. The fourth housing section 372 can include compartments 374a-f arranged in the same pattern as the holes 362a-f in the third housing section 358 and the compartments 350a-f in the second housing section 330. Each of the compartments 374a-f can accommodate a torque limiter 400 (shown in FIG. 18), which can be coupled to a respective output shaft 346a-f extending through a respective hole 362a-f. The fourth housing section 372 include holes 376a-f in end walls of the compartments 374a-f for passage of the connector shafts 402 of the torque limiters 400 outside of the fourth housing 372 when the rotatable assemblies 401 (shown in FIG. 18) of the torque limiters 400 are accommodated within the compartments 374a-f. The fourth housing section 372 can include fastening holes 378 that can be aligned with the fastening holes 370, 354, 326 in the housing sections 358, 330, 310. The compartments 374a-f can be arranged in a pattern to define a channel 375. The input shaft 324 can extend through the channel 375.

In some examples, as shown in FIGS. 22A, 22B, 22G, and 22H, the gearbox housing 304 can have a fifth housing section 380 disposed adjacent to the fourth housing section 372. The fifth housing section 380 can form a distal end portion of the gearbox housing 304. The fifth housing section 380 can include a base member 381 forming an end wall for the channel 375. A hole 382 can be formed in the base member 381 to allow the input shaft 324 to pass through the base member 381. The fifth housing section 380 can further include receptacles 384a-f formed in the base member 381 to receive end portions of the torque limiters 400 (shown in FIG. 18) when the rotatable assemblies 401 of the torque limiters 400 are disposed in the compartments 374a-f of the fourth housing section 372. The base member 381 can include openings 386a-f connected to the receptacles 384a-f such that the coupling sections 403 of the connector shafts 402 of the torque limiters 400 can be mounted in the openings 386a-f, or are accessible through the openings 386a-f, when the rotatable assemblies 401 are disposed in the compartments 374a-f in the fourth housing section 372. The fifth housing section 380 can include fastening holes 388 that can be aligned with the fastening holes 378, 370, 354, 326 in the housing sections 372, 358, 330, 310 to receive fasteners.

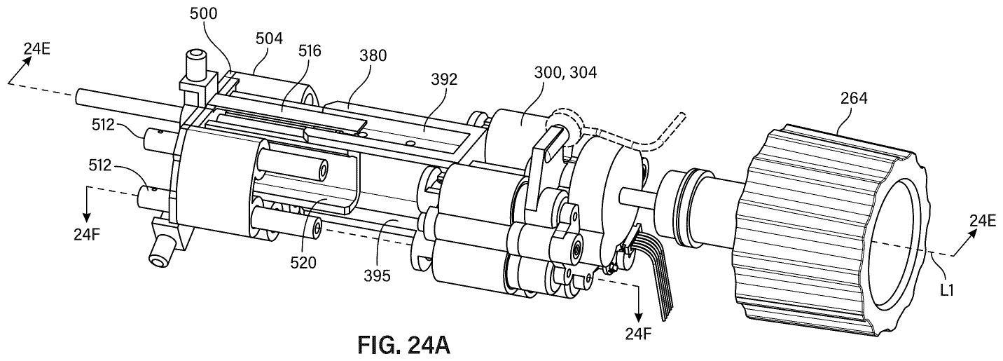

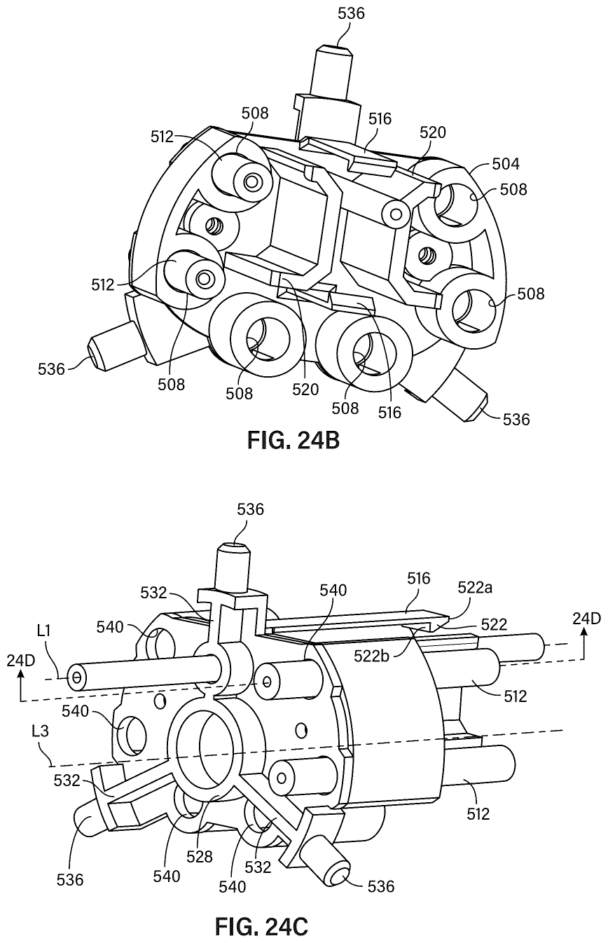

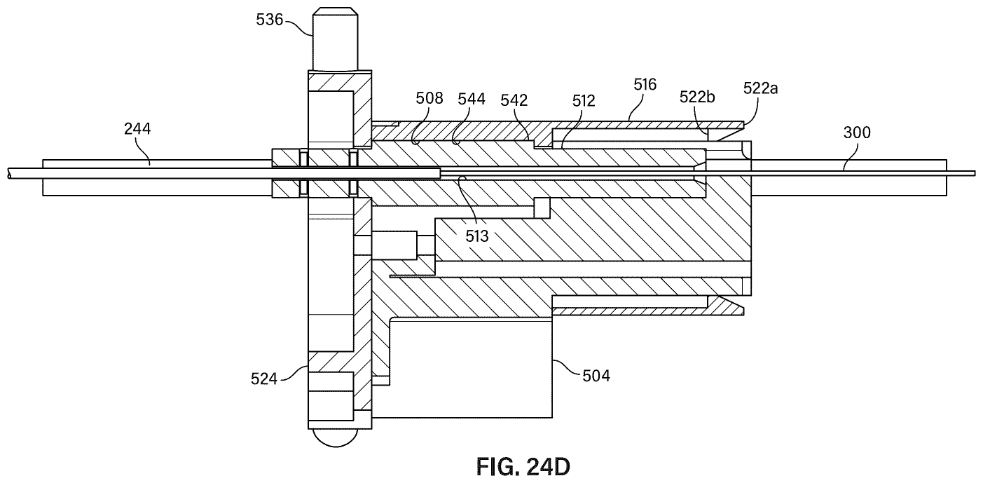

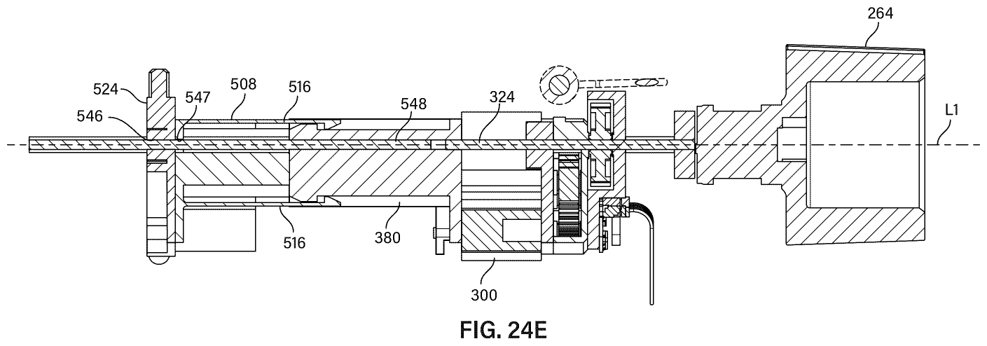

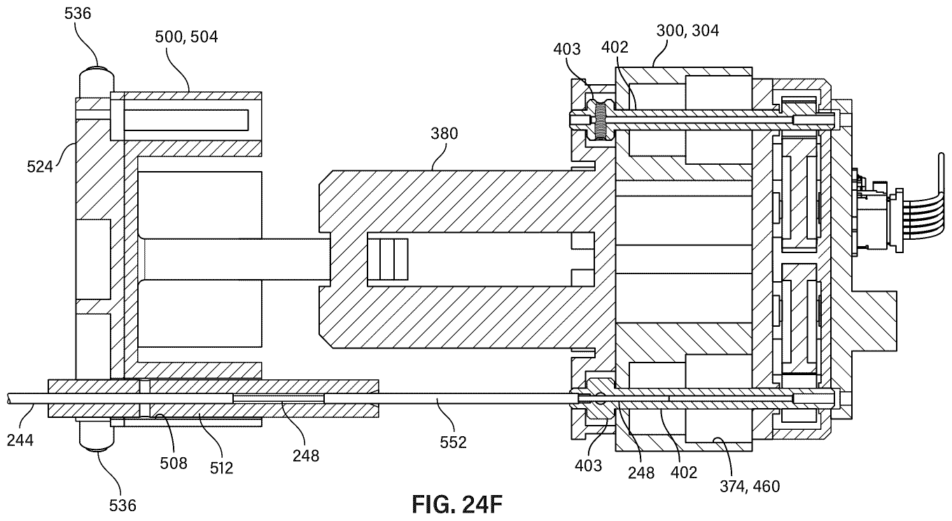

In some examples, as shown in FIGS. 22A, 22B, and 22H-22J, the fifth housing section 380 can further include a guide member 389 projecting from the base member 381. The guide member 389 can include a hole 390 aligned with the hole 382 in the plate member 381 to receive an end portion of the input shaft 324. Thus, when the gearbox 300 is fully assembled, the input shaft 324 will extend across all the housing sections 310, 330, 358, 372, and 380 (as shown in FIG. 24E). The input shaft 324 defines a longitudinal axis of the gearbox housing, which is also an axis about which the gearbox housing can pivot. The longitudinal axis of the gearbox housing 304 is aligned with the longitudinal axis L1 of the handle 204. A pair of guide slots 392 are formed on opposed surfaces (for example, top and bottom surfaces) of the guide member 389. The guide slots 392 extend axially in a direction along the longitudinal axis of the gearbox housing. Each guide slot 392 has opposed end walls 393, 394. A pair of guide channels 395 are formed on opposed sides of the guide member 389. The guide channels 395 extend axially in a direction along the longitudinal axis of the gearbox housing. As further described herein, the guide slots 392 and guide channels 395 can guide translation of a pull body along the longitudinal axis L1 of the handle.

The various housing sections 310, 330, 358, 372, and 380 of the gearbox housing 304 can be provided as separate members that are fastened together or as integral portions of the gearbox housing 304. In some cases, two or more of the housing sections 310, 330, 358, 372, and 380 can be integrally formed such that the gearbox housing 304 has fewer components to fasten together. In some cases, the gearbox housing 304 can be provided in two halves that can be fastened together. In other cases, the housing sections of the gearbox housing 304 can be attached together using means other than fasteners, for example, by welding, adhesive, and the like.

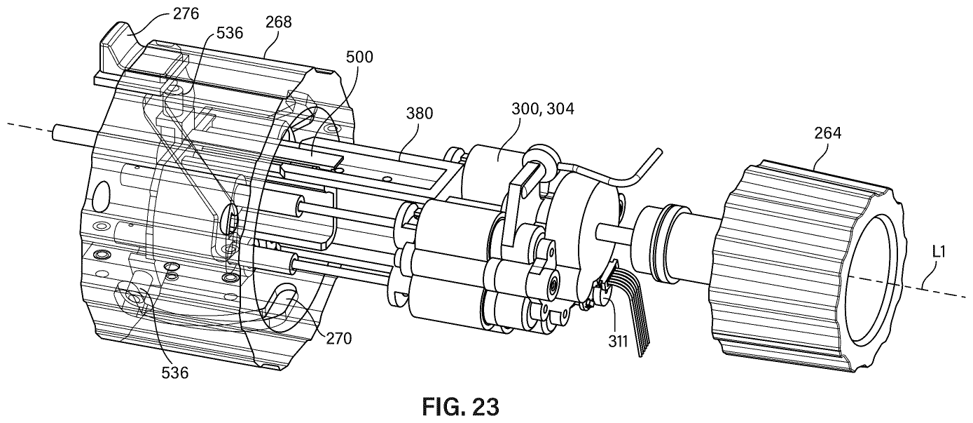

Referring to FIG. 23, the handle 204 can further include a pull body 500 disposed distally to the gearbox 300 and engaged with the distal end portion (or fifth housing section) 380 of the gearbox housing 304. The second knob 268 can rotatably engage the pull body 500 such that rotation of the second knob 268 relative to the handle body produces translation of the pull body 500 along the longitudinal axis L1 of the handle. The pull body 500 can be coupled to the outer sleeves 244 of the actuation assemblies 220 such that translation of the pull body 500 along the longitudinal axis L1 of the handle results in axial displacement of the outer sleeves 244 relative to the handle. This axial displacement can be used, for example, to axially displace the outer sleeves 244 relative to the corresponding actuator drivers 248 and thereby release the actuator drivers 248 from the prosthetic heart valve.

Referring to FIGS. 24A-24D, the pull body 500 has an axial axis L3 that is parallel to the longitudinal axis L1 of the handle. The pull body 500 includes a first pull body member 504 having a plurality of elongate sockets 508 axially aligned with the axial axis L3 of the pull body 500. Each of the sockets 508 can receive an actuation tube 512 (only two sockets 508 receiving actuation tubes 512 are illustrated in FIGS. 24A-24C). The number of sockets 508 can match the number of actuation assemblies 220 of the delivery apparatus. The sockets 508 are located on a side of the first pull body member 504 facing the gearbox 300.

The first pull body member 504 can include a pair of guide arms 516 extending in a direction parallel to the longitudinal axis L1 of the handle (and parallel to the axial axis L3 of the pull body) and towards the gearbox 300. The guide arms 516 are spaced in a direction transverse to the longitudinal axis L1 of the handle and are in opposed relation. Each guide arm 516 terminates in a hooked end 522. As illustrated in FIG. 24A, the guide arms 516 can be disposed in the respective guide slots 392 in the fifth housing section/distal end portion 380 of the gearbox housing 304. Each guide arm 516 can move within the respective guide slot 392 in a direction parallel to the longitudinal axis L1 of the handle. The hooked end 522 of the guide arm 516 has opposed surfaces 522a, 522b to engage the shoulders within the respective guide slot 392 in order to limit travel of the guide arm 516 in the proximal direction or the distal direction.

The first pull body member 504 can include a pair of guide members 520 extending in a direction parallel to the longitudinal axis L1 of the handle (and parallel to the axial axis L3 of the pull body 500). The guide members 520 are spaced in a direction transverse to the longitudinal axis L1 of the handle and are in opposed relation. As illustrated in FIG. 24A, the guide members 520 can be disposed in the respective guide channels 395 in the fifth housing section/distal end portion 380 of the gearbox housing 304. Each guide member 520 can move within the respective guide channel 395 in a direction parallel to the longitudinal axis L1 of the handle as the guide arms 516 move longitudinally within the respective guide slots 392.

The pull body 500 includes a second pull body member 524 disposed adjacent to the first pull body member 504. The second pull body member 524 can be attached to the first pull body member 524 by fasteners or other suitable method, such as welding, adhesive, and the like. The second pull body member 524 includes a central hub 528 having an axial axis that is aligned with the axial axis L3 of the pull body 500. The second pull body member 524 includes a plurality of radial arms 532 extending from the central hub 528 to a periphery of the pull body 500. The radial arms 532 are angularly spaced about the axial axis L3 of the pull body 500. Each radial arm 532 carries a pin 536 such that the pin 536 protrudes from the periphery of the pull body 500. The pins 536 are angularly spaced about the axial axis L3 of the pull body 500 by virtue of the radial arms 532 being angularly spaced about the axial axis L3 of the pull body 500.

The second pull body member 524 has a plurality of openings 540 corresponding in number and position to the plurality of sockets 508 in the first pull body member 504. The actuation tube 512 can thereby extend into the sockets 508 through the openings 540. As further illustrated in FIG. 24D, each actuation tube 512 can have keys 542 (for example, radial protrusions on the outer diameter of the actuation tube) that are received in slots 544 formed in the sockets 508 to prevent rotation of the actuation tube 512 within the sockets 508 (that is, the actuation tubes 512 are rotationally fixed relative to the pull body 500).

As illustrated in FIG. 24E, the first pull body member 504 and the second pull body member 524 have aligned openings 546, 547 to receive a guide rod 548. The guide rod 548 extends into an opening in the fifth housing section/distal end portion 380 of the gearbox housing 304 and together with the guide arms 516 and guide members 520 maintain longitudinal alignment of the pull body 500 with the gearbox 300 as the guide arms 516 move within the respective slots 392 in the fifth housing section/distal end portion 380 of the gearbox housing 304.

As illustrated in FIGS. 24D and 24F, the lumen 513 of each actuation tube 512 receives a proximal end portion of an outer sleeve 244 of an actuation assembly 220. The outer sleeve 244 is fixedly attached to the actuation tube 512 (for example, using set screws). In some cases, the actuation tube 512 may not extend all the way to the coupling section 403 disposed at the distal end of the gearbox 300 (for example, the coupling section 403 disposed adjacent to a compartment 374 or housing 460 that holds the rotatable assembly 401 of the torque limiter 400 as depicted in FIG. 18). In these cases, to provide support to the proximal end portion of the actuation tube 512, a support extension tube 552 can extend from the coupling section 403 into the lumen 513 of the actuation tube 512. The support extension tube 552 can be fixedly attached to the coupling section 403. The support extension tube 552 is not fixed to the actuation tube 512 so that the actuation tube 512 can translate over the support extension tube 552 towards or away from the coupling section 403 as the guide arms 516 move along the slots 392 in the fifth housing section/distal end portion 380 of the gearbox 304. Each actuation driver 248 extends through the respective outer sleeve 244, through the lumen 513 of the actuation tube 512, through the support extension tube 552, into the connector shaft 402 and is secured to the connector shaft 402.

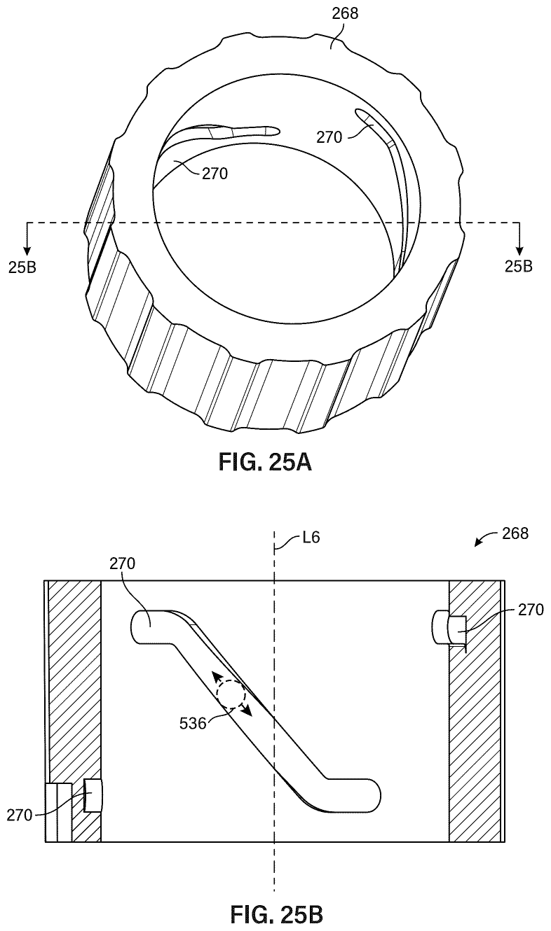

Referring to FIGS. 25A and 25B, inner channels 270 are formed along the inner surface of the second knob 268. The number of inner channels 270 can match the number of pins 536 of the pull body 500. The number of inner channels 270 can be angularly spaced about a central axis L6 of the second knob 268 such that when the second knob 268 is disposed around the pull body 500 (as illustrated in FIG. 23), each pin 536 can extend into a corresponding inner channel 270. Each channel 270 can have a Z shape (or S shape) as depicted in FIG. 25B.

When the second knob 268 is rotated, the pins 536 slide along the inner channels 270. As the pins 536 slide along the inner channels 270, the pull body 500 is translated along the longitudinal axis L1 of the handle. To release the actuation assemblies 220 from the prosthetic heart valve 100 (for example, after radially expanding the prosthetic heart valve 100 at the implantation location), the second knob 268 can be rotated in a direction to move the pull body 500 proximally (that is, towards the gearbox 300). Since the outer sleeves 244 are attached to the pull body 500, the outer sleeves 244 are axially displaced in a direction along the longitudinal axis L1 of the handle. The axial displacement of the outer sleeves 244 can retract the outer sleeves 244 from the frame 104 and from the flexible elongated elements 254, allowing the flexible elongated elements 254 (shown in FIGS. 7A-7B) of the actuator drivers 248 to be released from the actuator heads 176 of the prosthetic heart valve 100.

During expansion of the prosthetic heart valve 100, rotational movement of the actuator drivers 248 by operation of the gearbox 300 applies a torque to the prosthetic heart valve 100 that tends to rotate the prosthetic heart valve about the longitudinal axis L of the prosthetic heart valve. Since the outer sleeves 244 are engaged with the frame 104 of the prosthetic heart valve 100, the outer sleeves 244 tend to rotate around the longitudinal axis L of the prosthetic heart valve 100. Since the pull body 500 is coupled to the outer sleeves 244, the pull body 500 likewise tends to rotate with the outer sleeves 244.

The gearbox 300 can pivot about the longitudinal axis L1 of the handle, which is aligned with the axial axis of the input shaft 324 and the axial axis of the guide rod 548. Thus, rotation of the pull body 500 during expansion of the prosthetic heart valve 100 can result in pivoting of the gearbox 300 about the longitudinal axis L1 of the handle 204. In some examples, the handle 204 includes a mechanism to limit pivoting of the gearbox 300 at least during expansion of the prosthetic heart valve 100. In some examples, the mechanism can include a stop member that engages the gearbox housing 304 when the gearbox housing 304 is in a predetermined rotational position relative to the body of the handle 204.

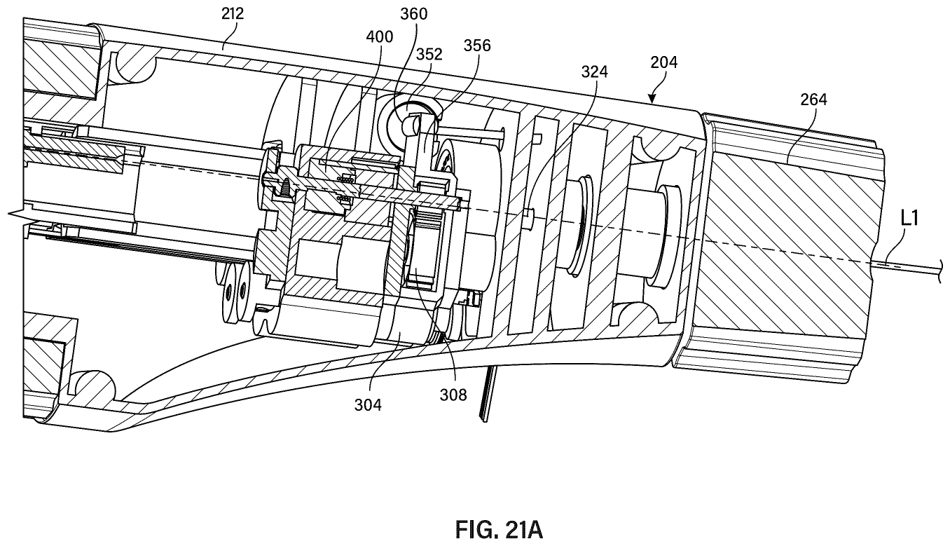

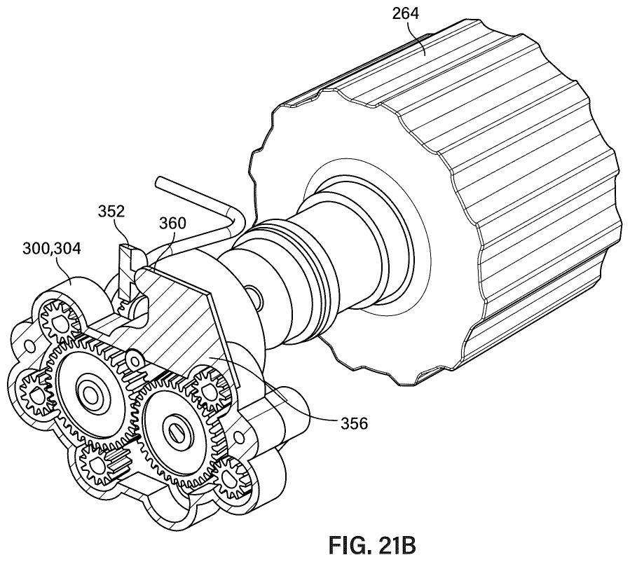

Referring to FIGS. 21A, 21B, 22C-22E, the gearbox housing 304 can have an extension arm 356 projecting from an outer surface of the gearbox housing 304. When the gearbox housing 304 is positioned within the cavity 205 of the handle 204, the extension arm 356 extends into a portion of the cavity 205 surrounding the gearbox housing 304 (as shown in FIG. 21A). In some examples, the extension arm 356 can be a flat member lying in a plane transverse to the longitudinal axis L1 of the handle. A protrusion member 360 can be attached to or integrally formed with the extension arm 356. The protrusion member 360 can be in the form of a rod or pin. The protrusion member 360 can have a rounded end 359 for contact with a stop member. The protrusion member 360 can be oriented in a direction transverse to the longitudinal axis L1 of the handle. The extension arm 356 can position the protrusion member 360 such that an axial axis L4 (depicted in FIG. 22E) of the protrusion member 360 is tangential to a circular path 361 centered around the pivoting axis of the gearbox 300 (or longitudinal axis L1 of the handle). This could also be described as the protrusion member 360 being radially outward of the pivoting axis of the gearbox 300. Thus, the protrusion member 360 moves along the circular path 361 as the gearbox 300 pivots.

The extension arm 356 is shown as an integral part of the housing section 330 of the gearbox housing 304. However, the extension arm 356 could be an integral part of any of the other housing sections of the gearbox housing in other examples. Also, the extension arm 356 is shown at the top of the housing section 330. However, it could be located elsewhere on the housing section 330 provided that it positions the protrusion member 360 along the circular path 361. Alternatively, the circular path can be larger or smaller than the circular path 361 so long as it is coaxial with the longitudinal axis L1 of the handle.

A stop member 352 can be mounted to an inner surface of the proximal body portion 212 of the handle 204 (as depicted in FIG. 21A) such that the protrusion member 360 can contact the stop member 352 as the protrusion member 360 moves along the circular path 361. The stop member 352 can be positioned such that an axial axis L5 (shown in FIG. 22E) of the stop member 352 is also tangential to the circular path 361. Thus, as the protrusion member 360 moves along the circular path 361, the protrusion member 360 will encounter the stop member 352. In some examples, the stop member 352 can be positioned such that when the first knob 264 (shown in FIG. 21A) is rotated in a direction to expand the prosthetic heart valve (for example, in the clockwise direction when viewing from the proximal end of the handle), the stop member 352 acts to limit the pivoting of the gearbox 300.

In some examples, the first knob 264 can be rotated in a direction to expand the prosthetic heart valve 100 (for example, in the clockwise direction when viewing from the proximal end of the handle). As the prosthetic heart valve 100 is expanded, if the protrusion member 360 is not yet in contact with the stop member 352, the entire gearbox 300 can pivot about the longitudinal axis L1 of the handle (which is the same as the axial axis of the input shaft 324 as depicted in FIG. 21A) in a direction towards the stop member 352. As the gearbox 300 pivots, the protrusion member 360 moves along the circular path 361 until the protrusion member 360 encounters the stop member 352, which then prevents further pivoting of the gearbox 300 in the same direction. While the protrusion member 360 is in contact with the stop member 352, the gear train 308 can still be operated through rotation of the first knob 264 and input shaft 324.

In some examples, the stop member 352 can be a load cell (or force sensor) such that when the protrusion member 360 is in contact with the stop member 352 during expansion of the prosthetic heart valve (as depicted more clearly in FIG. 21B; the stop member 352 is shown in FIG. 21B without the handle body to which it is coupled for simplicity of illustration), any load applied to the stop member 352 by the protrusion member 360 can be measured. This measured load can be used to determine the torque applied to the prosthetic heart valve 100 during expansion of the valve. For example, the measured load can be multiplied by the moment arm as defined by the extension arm 356. Thus, the stop member 352 implemented with a load cell can perform the function of limiting pivoting of the gearbox 300 and measuring torque applied to the prosthetic heart valve 100. Load cells can be provided in dimensions that are significantly smaller than those of conventional torque meters, allowing a more compact handle design.

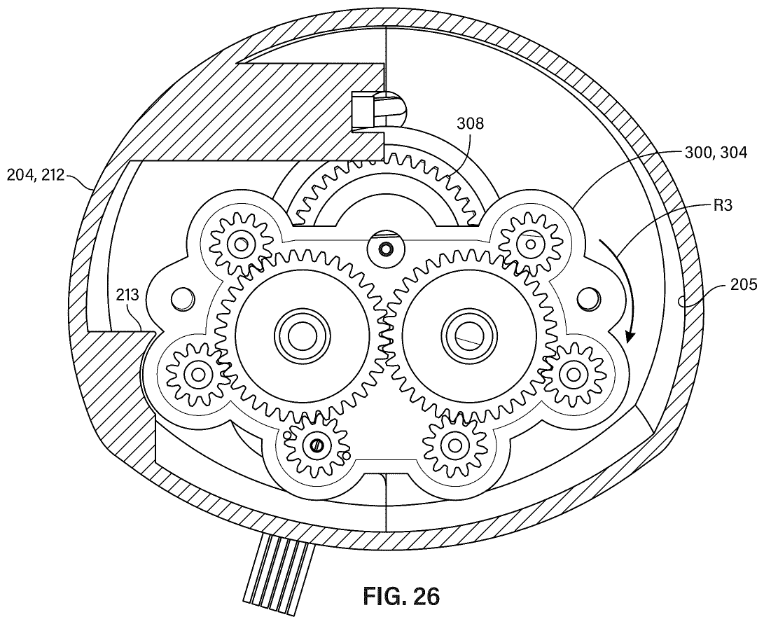

As the first knob 264 is rotated in a direction that compresses the prosthetic heart valve 100 (for example, in the counterclockwise direction when viewing from the proximal end of the handle), the protrusion member 360 is spaced away from the stop member 352. As such, the stop member 352 does not act to limit pivoting of the gearbox 300 and does not measure torque when the prosthetic heart valve 100 is being compressed. In some cases, the handle body can act to limit pivoting of the gearbox 300 during compression of the prosthetic heart valve 100. For example, as illustrated in FIG. 26, the proximal body portion 212 of the handle 204 can include an inner protrusion 213 that engages the gearbox 300 when the gearbox 300 pivots in a direction R3 corresponding to compression of the prosthetic heart valve (for example, the counterclockwise direction when viewed from the proximal end of the handle or the clockwise direction when viewed from the distal end of the handle). In some cases, the gearbox 300 can be provided with a second extension arm and protrusion member, and a second load cell (or a multi-directional load sensor) can be mounted on the handle body. The protrusion member on the second extension arm can be arranged to contact the second load cell to measure the torque applied to the prosthetic heart valve 100 while compressing the prosthetic heart valve.

Referring to FIGS. 1-26, the prosthetic heart valve 100 can be placed in a radially compressed configuration, and the actuation assemblies 220 of the delivery apparatus 200 can be releasably coupled to the actuators 168 of the prosthetic heart valve 100. The delivery apparatus 200 and the prosthetic heart valve 100 can be advanced over a guidewire through the vasculature of a patient to a selected implantation location (for example, the native aortic annulus). For example, when implanting the prosthetic heart valve 100 within the native aortic valve, the delivery apparatus 200 and the prosthetic heart valve 100 can be inserted into and through a femoral artery, and through the aorta to the native aortic valve. The prosthetic heart valve 100 can then be deployed at the implantation location.

In some examples, the prosthetic heart valve 100 is enclosed in a delivery capsule 226 prior to insertion into the patient's vasculature. In this case, the third knob 272 can be operated to retract the delivery capsule 226 and expose the prosthetic heart valve 100. To deploy the prosthetic heart valve 100, the physician can turn the first knob 264 to rotate the set of first actuator drivers (for example, 248a, 248b, 248c) in a first direction and the set of second actuator drivers (for example, 248d, 248e, 248f) in a second direction, corresponding to counter-rotation of the first and second sets of the actuators of the prosthetic heart valve 100 in a direction that radially expands the prosthetic heart valve 100.

During the valve expansion, the torque exerted on the native anatomy can be measured via the stop member/load cell 352 in the handle 204. During the valve expansion, torque limiter(s) 400 can stop the gearbox 300 if respective actuator driver(s) 248 become overloaded. After the prosthetic heart valve 100 has been expanded to the working diameter by rotation of the actuators, the actuation assemblies 220 can be released from the prosthetic heart valve 100. To release the actuation assemblies 220, the pull body 500 can be translated proximally along the longitudinal axis L1 of the handle 204 (for example, by rotating the second knob 268) so as the retract the outer sleeves 244 from the frame 104 of the prosthetic heart valve 100 and the flexible elongated elements 254 of the actuator drivers 248. The freed flexible elongated elements 254 can be removed from the actuator heads 176 of the prosthetic heart valve 100, allowing the delivery apparatus to be withdrawn from the body.

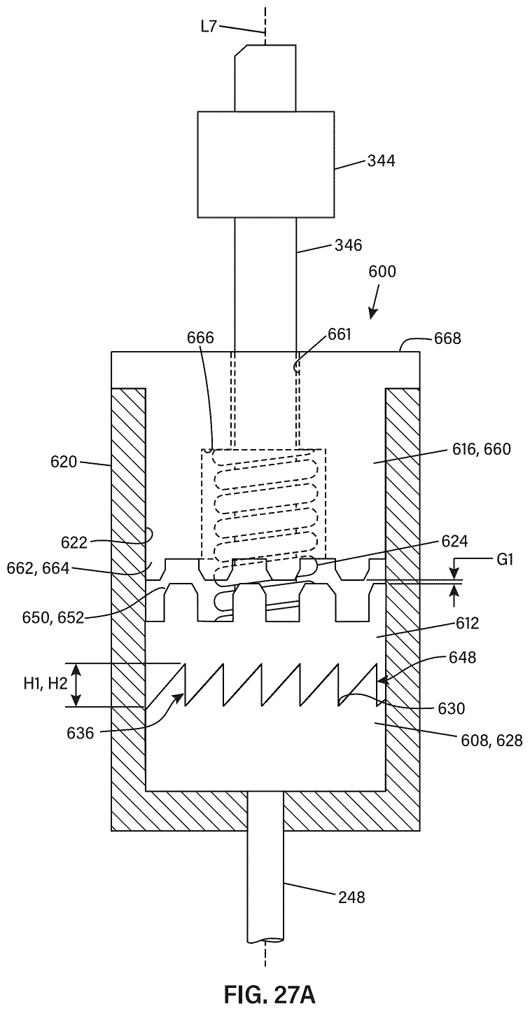

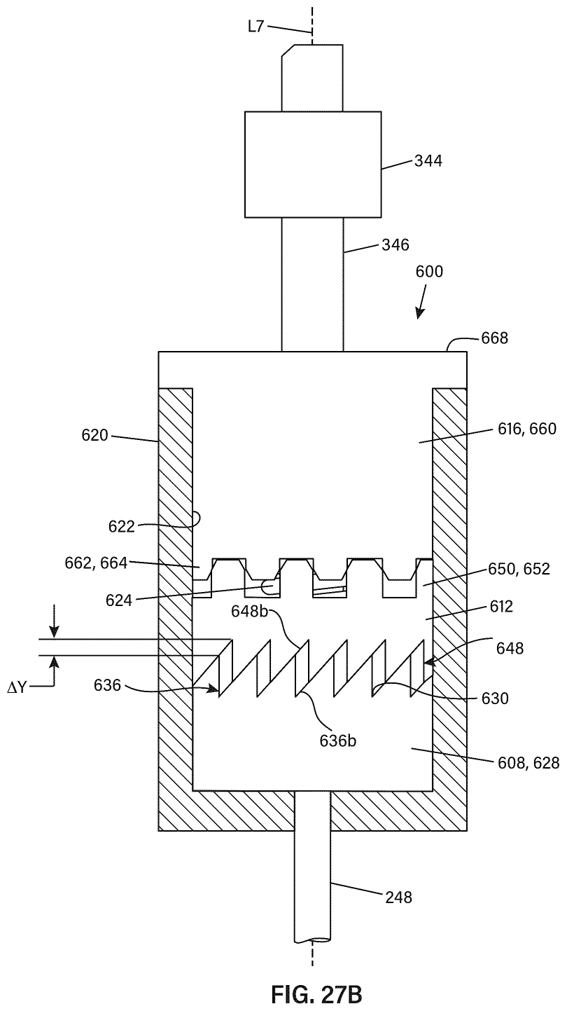

FIGS. 27A and 27B illustrate a torque limiter 600, according to some examples. The torque limiter 600 can couple an actuator driver 248 to an output shaft 346 of the gearbox 300 (shown in, for example, FIGS. 9A-9C) and can operate to prevent rotation of the actuator driver 248 when a torque on the actuator driver 248 exceeds a threshold. In the unlocked state, as shown in FIG. 27A, the torque limiter 600 transfers rotation of the output shaft 346 to the actuator driver 248. In the locked state, as shown in FIG. 27B, rotation of the actuator driver 248 via the output shaft 346 is prevented. The torque limiter 600 transitions from the unlocked state to the locked state when the torque on the actuator driver 248 exceeds the threshold, as further explained below. The threshold can be a maximum torque allowable on the actuator driver 248 (or within a tolerance of the maximum torque allowable on the actuator driver 248) while rotating the actuator driver 248 in order to rotate the prosthetic heart valve. Continued rotation of the actuator driver 248 at a torque above the maximum torque may result in damage to the actuator driver 248 and/or prosthetic heart valve. Transitioning the torque limiter 600 to the locked state when the torque on actuator driver 248 exceeds the threshold prevents continued operation of the actuator driver 248 above the maximum torque.

The torque limiter 600 includes a housing 620 having a longitudinal axis L7. The housing 620 can be a separate housing that can be attached to the gearbox 300 or can be an integral compartment of the gearbox housing 304 (shown, for example, in FIGS. 9A-C).

The torque limiter 600 includes a driver member 608, an engagement member 612, and a base member 616. The driver member 608, engagement member 612, and base member 616 can, in some examples, be disposed within a chamber 622 of the housing 620 and axially aligned along the longitudinal axis L7. The engagement member 612 is fixedly secured to the output shaft 346 and is situated between the driver member 608 and the base member 616 and is axially movable along the longitudinal axis L7. The driver member 608 is fixedly secured to the actuator driver 248. The driver member 608 and the engagement member 612 are rotatable relative to the housing 620 and about the longitudinal axis L7. The base member 616 is rotationally fixed relative to the longitudinal axis L7 (for example, via the housing 620).

The actuator driver 248 is attached to the driver member 608 and rotates with the driver member 608. The output shaft 346 is attached to the engagement member 612 and rotates with the engagement member 612. The output shaft 346 can extend from the engagement member 612, through a bore in the base member 616, and to a gear 344. The gear 344 is part of a gear train (for example, the gear train 308 shown in FIGS. 10A-10C).

In the unlocked state of the torque limiter 600 (as shown in FIG. 27A), the engagement member 612 engages the driver member 608 and is separated from the base member 616 (for example, by a gap G1). In the unlocked state, torque can be transferred, for example, from a handle, through the gearbox, to the output shaft 346, and to the actuator driver 248 through the engagement member 612 and driver member 608. When the torque generated by the actuator driver 248 (for example, on the engagement member 612 via the rotation of the driver member 608) exceeds a threshold, the engagement member 612 can be axially displaced towards the base member 616 until the engagement member 612 engages the base member 616 (as shown in FIG. 27B). At this point the torque limiter 600 is in the locked state (or locked), and the engagement member 612 can no longer be rotated with respect to the driver member 608. Since the output shaft 346 is connected to the engagement member 612, rotation of the output shaft 346 is also prevented when the torque limiter is in the locked state.

In some example, due to the connection between the gears of the gearbox, all actuator drivers are prevented from rotating when one or more of the torque limiters is locked. A locked torque limiter also prevents rotation of the actuation knob. In this manner, in some examples, a handle for a delivery apparatus can include one or more torque limiters regardless of whether the delivery apparatus comprises one or more actuator drivers. In some examples, a handle for a delivery apparatus can comprise a plurality of torque limiters (for example, one torque limiter for each actuator driver).

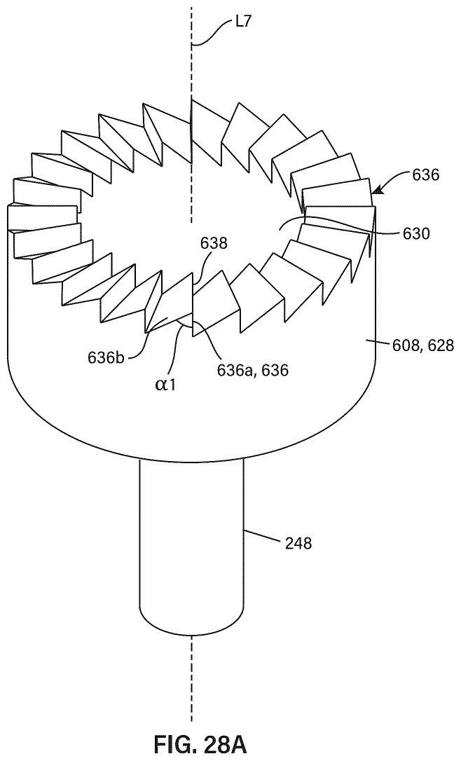

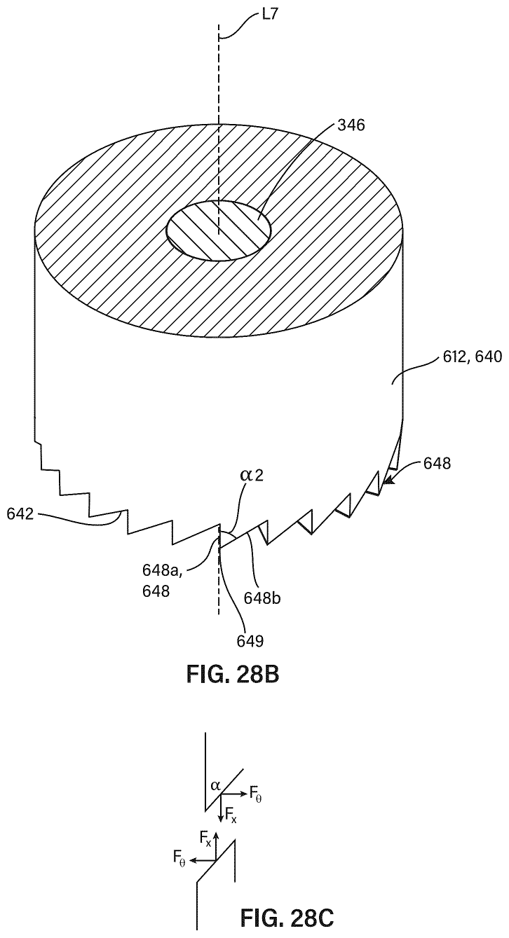

Referring to FIG. 28A, the driver member 608 of the torque limiter 600 can include a driver body 628. A proximal end face 630 of the driver body 628 includes a set of driver teeth 636 disposed along an axis perpendicular to the longitudinal axis L7. Each driver tooth 636 in the set of driver teeth 636 has opposed tooth surfaces 636a, 636b. In some examples, the tooth surface 636a is an axial surface extending in an axial direction defined by the longitudinal axis L7 (for example, parallel to the longitudinal axis L7), and the tooth surface 636b is an inclined surface (for example, inclined to the tooth surface 636a and the axial direction). In other examples, the tooth surface 636 may be an inclined surface (for example, not extending parallel to the longitudinal axis L7). The tooth surfaces 636a, 636b are joined at a tooth apex 638 having an angle α1.

The actuator driver 248 extends distally from a distal end of the driver body 628. The actuator driver 248 can be coupled to the driver body 628 using any suitable method (such as by inserting an end portion of the actuator driver 248 into a bore in the driver body 628 and securing the end portion in the bore via adhesive, fasteners, and/or other means for coupling). In some examples, the actuator driver 248 and the driver member 608 can be integrally formed as a single, unitary component.

Referring to FIG. 28B, the engagement member 612 includes an engagement member body 640. A distal end face 642 of the engagement member body 640 includes a set of engagement teeth 648 arranged about the longitudinal axis L7. Each engagement tooth 648 in the set of engagement teeth 648 has opposed tooth surfaces 648a, 648b. In some examples, the tooth surface 648a is an axial surface extending in the axial direction defined by the longitudinal axis L7 (for example, parallel to the longitudinal axis L7), and the tooth surface 648b is an inclined surface (for example, inclined to the tooth surface 648a and the axial direction). In some examples, the tooth surface 648a may be an inclined surface (for example, not extending parallel to the longitudinal axis L7). The tooth surfaces 648a, 648b are joined at a tooth apex 649 having an angle α2. In some examples, the angle α2 can be the same as the angle α1 (shown in FIG. 28A). In other examples, the angle α2 can be different than the angle α1. The inclined tooth surfaces 636b, 648b can slide relative to each other as the engagement member 612 is axially displaced relative to the driver member 608.

The output shaft 346 extends proximally from the proximal end of the engagement member body 640. The output shaft 346 can be coupled to the engagement member body 640 using any suitable method (such as by inserting an end portion of the output shaft 346 into a bore in the engagement member body 640 and securing the end portion in the bore via adhesive, fasteners, and/or other means for coupling). In some examples, the output shaft 346 and the engagement member 612 can be integrally formed as a single, unitary component.

As shown in FIGS. 27A and 27B, in the torque limiter 600, the engagement member 612 is axially aligned with the driver member 608 and oriented such that the set of engagement teeth 648 is in opposing relation to the set of driver teeth 636. In some examples, the set of engagement teeth 648 can be complementary to the set of driver teeth 636 in that the set of engagement teeth 648 can engage with the set of driver teeth 636, as shown in FIGS. 27A and 27B. In some examples, the tooth profile of the engagement teeth 648 can be the same as the tooth profile of the driver teeth 636 (for example, the angles α1 are α2 the same, and the slope of the tooth surfaces of the engagement teeth 648 and the driver teeth 636 are the same). In some examples, the tooth profile of the engagement teeth 648 can be different from the tooth profile of the driver teeth 636 (for example, the angles α1 are α2 can be different, or the angles α1 are α2 can be the same but the slope of the tooth surfaces of the engagement teeth 648 and the driver teeth 636 can be different).

In some examples, the torque limiter 600 can include a biasing member, which is illustrated as a spring 624, but can be a different type of biasing member (such as, for example, an elastically deformable member, a hydraulic piston, a pneumatic piston, etc.). The spring 624 (or biasing member) is configured to bias the engagement member 612 away from the base member 616 and against the driver member 608. In the unlocked state of the torque limiter 600, the spring 624 (or biasing member) can bias the engagement member 612 against the driver member 608 such that the set of engagement teeth 648 fully engages the set of driver teeth 636 (see, for example, FIG. 27A). When torque on the actuator driver 248 exceeds a threshold, the engagement member 612 is moved axially away from the driver member 608 and the spring 624 (or biasing member) is compressed (see, for example, FIG. 27B).

FIG. 28C illustrates force components at an interface between an opposing driver tooth 636 and engagement tooth 648. The force component Fₓ acts in a direction parallel to the longitudinal axis L7 (or in the axial direction), and the force component F_{θ} acts in a direction transverse to the longitudinal axis L7. The corresponding axial and tangential forces between the teeth 636, 648 can be expressed as follows:

$${F}_{θ} = M / R \qquad ( 1 )$$

$${F}_{x} = K Δ Y \qquad ( 2 )$$

$${F}_{T} ( α ) = {F}_{x} / {F}_{θ} \qquad ( 3 )$$

In Equations (1)-(3), Fₓ is a force component acting on the teeth in a direction parallel to the longitudinal axis L7 (or in the axial direction), F_{θ} is a force component acting on the teeth in a direction transverse to the longitudinal axis L7, M is moment applied to the teeth by the actuator driver, R is the moment arm (for example, the radial distance of the teeth from the longitudinal axis L7), F_{T} is the tangential force applied to the teeth, α is the angle between the tooth surfaces, k is the spring constant of the spring 624 (or bias constant of the biasing member), and ΔY is the distance by which the engagement member 612 (or the set of engagement teeth 648) is displaced from the driver member 608 (or from the set of driver teeth 636). When the sets of teeth 636, 648 are fully engaged (as shown in FIG. 27A), ΔY is zero.

Returning to FIGS. 27A and 27B, a proximal end face 650 of the engagement member 612 includes a set of locking teeth 652. The base member 616 includes a base body 660. A distal end face 662 of the base body 660 includes a set of locking teeth 664 that is positioned in opposing relation to the set of locking teeth 652 of the engagement member 612 and can interlock with the set of locking teeth 664. In the unlocked state of the torque limiter 600, the set of locking teeth 652 of the engagement member 612 is separated from the set of locking teeth 664 of the base member 616 by a gap G1 (shown in FIG. 27A). This separation can be maintained by the spring 624 that biases the engagement member 612 towards the driver member 608. While the locking teeth 664, 652 are separated, the engagement member 612 can be rotated about the longitudinal axis L7 by the output shaft 346.

The base body 660 can include a proximal flange 668 that can be used to attach the base member 616 to the housing 620, which would prevent rotation of the base member 616 relative to the housing 620 and about the longitudinal axis L7. The output shaft 346 can extend proximally from the engagement member 612 and through a central opening or bore 661 in the base body 660. The spring 624 is disposed around a portion of the output shaft 346 between the engagement member 612 and the base member 616. One end of the spring 624 can be attached to the engagement member 612, while the other end of the spring 624 bears against a surface 666 of the base member 616 (or vice versa).

In the unlocked state of the torque limiter 600, the spring 624 is in the free state and biases the engagement member 612 towards the driver member 608. In the unlocked state, the set of locking teeth 652 of the engagement member 612 is separated from the set of locking teeth 664 of the base member 616 by a gap G1, which allows the engagement member 612 (and the output shaft 346) to rotate freely relative to the base member 616. The biasing force of the spring 624 can be overcome when the torque on the actuator driver 248 exceeds a threshold. When this occurs, the engagement member 612 can be displaced axially towards the base member 616 until the set of locking teeth 652 of the engagement member 612 engages and interlocks with the set of locking teeth 664 of the base member 616, as shown in FIG. 27B. Since the base member 616 is rotationally fixed, rotation of the engagement member 612 will be prevented when the set of locking teeth 652 of the engagement member 612 is interlocked with the set of locking teeth 664 of the driver member 616. As a result, the driver member 608 and the actuator driver 248, as well as the output shaft 346, are prevented from rotating relative to the base member 616. This can, for example, reduce the likelihood of the actuation shafts of the prosthetic heart valve from being damaged.

In some examples, the angle α (corresponding to α1 and α2 in FIGS. 28A and 28B) between the tooth surfaces of the driver tooth 636 and the engagement tooth 648 and the spring constant k of the spring 624 can be selected to allow displacement ΔY of the engagement member 612 in response to a torque M on the actuator driver 248 such that when the torque M exceeds the threshold, the displacement ΔY of the engagement member 612 is sufficient to interlock the set of locking teeth 652 of the engagement member 612 with the set of locking teeth 664 of the base member 616, which would stop further rotation of the engagement member 612. The threshold can be within a tolerance of a maximum torque that can be on the actuator driver 248 (for example, within 15% of the maximum torque). In some examples, the maximum torque on the actuator driver 248 can be 50 N-mm.

When the engagement member 612 stops rotating due to interlocking of the sets of teeth 652, 664, the output shaft 346 and the gear 344 coupled to the output shaft 346 will stop rotating. Since all the gears within the gearbox 300 are interconnected (as illustrated, for example, in FIG. 10A), the entire gearbox will halt as well, which would prevent rotational movement of all the other actuator drivers coupled to the gearbox 300. Each of the actuator drivers coupled to the gearbox 300 can be provided with a torque limiter 600 so that the gearbox 300 can be halted once the torque on any one of the actuator drivers exceeds the threshold.

In some examples, as illustrated in FIG. 27A, the axial lengths H1, H2 (shown in FIG. 27A) of the set of engagement teeth 648 and the set of driver teeth 636 can be longer than the gap G1 (shown in FIG. 27A) between the set of locking teeth 652 and the set of locking teeth 664 in the unlocked state of the torque limiter 600. Thus, when the gap G1 is closed during interlocking of the sets of locking teeth 652, 664 in the locked state of the torque limiter 600, as illustrated in FIG. 27B, the inclined tooth surfaces 648b of the set of engagement teeth 648 can remain engaged with the inclined tooth surfaces 636b of the set of driver teeth 636. While the sets of engagement teeth 648 and driver teeth 636 are engaged with each other and the sets of locking teeth 652, 664 are interlocked with each other, rotation of the actuator driver 248 will be prevented.

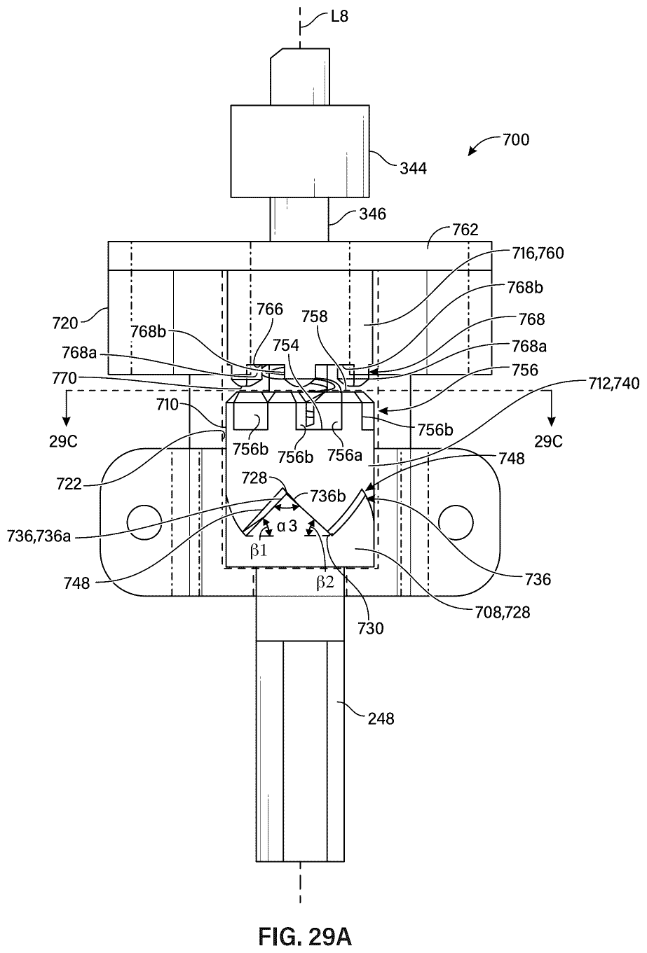

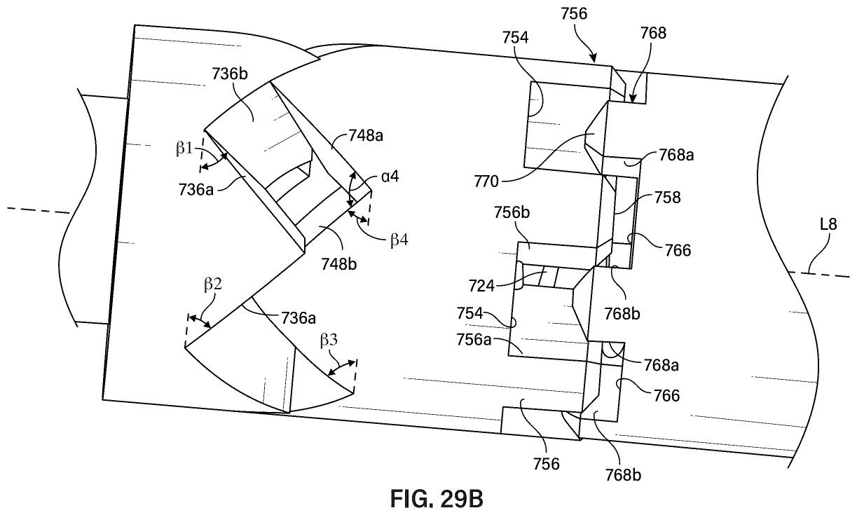

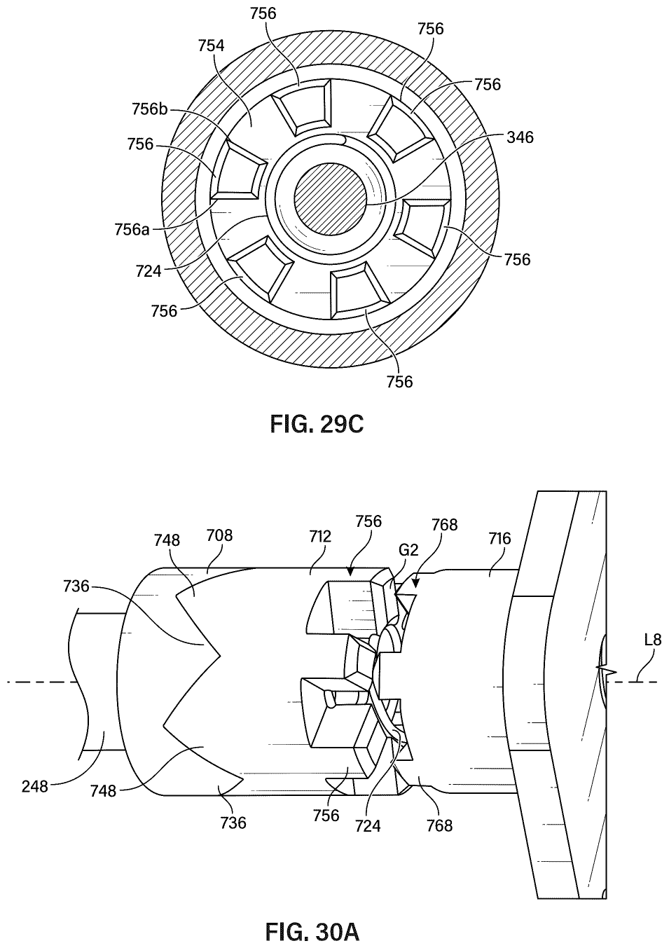

FIG. 29A illustrates a torque limiter 700, according to some examples. In a similar manner to the torque limiter 600 described herein, the torque limiter 700 includes a driver member 708, an engagement member 712, and a base member 716 disposed within a cavity 722 of a housing 720 having a longitudinal axis L8. The members 708, 712, 716 are axially aligned along the longitudinal axis L8. The engagement member 712 is situated between the driver member 708 and the base member 716. The driver member 708 and the engagement member 712 are rotatable relative to the housing 720 and about the longitudinal axis L8. The base member 716 is rotationally fixed relative to the housing 720 and about the longitudinal axis L8.

The driver member 708 includes a driver body 728. A proximal end face 730 of the driver body 728 includes a set of driver teeth 736. The driver teeth 736 are disposed about the longitudinal axis L8, with the roots of adjacent teeth 736 connected to each other at the proximal end face 730. Each driver tooth 736 has opposed tooth surfaces 736a, 736b. In some examples, the tooth surfaces 736a, 736b are inclined relative to the longitudinal axis L8. The tooth surfaces 736a, 736b are inclined towards each other and joined at a tooth apex having an acute angle α3. The inclination angles β1 and β2 of the tooth surfaces 736a, 736b can be different.

The actuator driver 248 extends distally from a distal end of the driver body 728. The actuator driver 248 can be coupled to the driver body 728 using any suitable method (such as by inserting an end portion of the actuator driver 248 into a bore in the driver body 728 and securing the end portion in the bore).

The engagement member 712 includes an engagement body 740. A distal end face of the engagement body 740 includes a set of engagement teeth 748 arranged about the longitudinal axis L8. As shown in FIG. 29B, each engagement tooth 748 has opposed tooth surfaces 748a, 748b. In some examples, the tooth surfaces 748a, 748b are inclined relative to the longitudinal axis L8 and towards each other. The tooth surfaces 748a, 748b form a tooth apex 750 having an angle α4, which can be the same as or different than angle α3 between the driver tooth surfaces 736a, 736b. The inclination angles β3 and β4 of the tooth surfaces 748a, 748b can be the same as or different than inclination angles β1 and β2 of the driver tooth surfaces 736a, 736b.

Returning to FIG. 29A, the output shaft 346 extends proximally from the proximal end of the engagement member 740. The output shaft 346 can be coupled to the engagement member body 740 using any suitable method (such as by inserting an end portion of the output shaft 346 into a bore in the engagement member body 740 and securing the end portion in the bore).

The engagement member 712 is axially aligned with the driver member 708 and oriented such that the set of engagement teeth 748 is in opposing relation to the set of engagement teeth 732. The sets of teeth 732, 744 are complementary in that the teeth 732,744 can engage each other in both the locked and unlocked states of the torque limiter. In some examples, the teeth 732, 744 can be complementary and have the same tooth profile. In other examples, the teeth 732, 744 can be complementary and have different tooth profiles. The two inclined surfaces of each of the teeth 736, 748 allow the sets of teeth 732, 744 to slide over each other during rotational movement of the actuator driver 248 in either direction.

A proximal end portion 710 of the engagement member 712 includes a set of locking teeth 756 disposed about the longitudinal axis L8. The locking teeth 756 in the set of locking teeth 756 are angularly spaced apart about the longitudinal axis L8 by slots 754. Each locking tooth 756 has opposed tooth surfaces 756a, 756b radially oriented relative to the longitudinal axis L8. The opposed tooth surfaces 756a, 756b are connected to a tooth land 758. The edges between the tooth surfaces 756a, 756b and the tooth top 758 can be chamfered. Tooth surfaces 756a, 756b on adjacent teeth 756 can be angled to form a wedge-shaped slot 754 (shown in FIG. 29C) between the teeth.

The base member 716 includes a base body 760 having a distal end in opposing relation to a proximal end of the engagement member 712. A distal end portion of the base body 760 includes a set of locking teeth 768, which is complementary to the set of locking teeth 756 in that the locking teeth 768 can engage (for example, mesh) with the locking teeth 756. The locking teeth 768 in the set of locking teeth 768 are spaced apart by slots 766. Each locking tooth 768 has opposed tooth surfaces 768a, 768b radially oriented relative to the longitudinal axis L8. The opposed tooth surfaces 768a, 768b are connected to a tooth land 770. The edges between the tooth surfaces 768a, 768b and the tooth top 770 can be chamfered. Tooth surfaces 768a, 768b on adjacent teeth 768 can be angled to form a wedge-shaped slot 754 (shown in FIG. 29C) between the teeth.

To form an interlock between the sets of teeth 756, 768, the engagement member 712 can be displaced toward the base member 716 until the tooth tops 758, 770 are proximate each other. The engagement member 712 can be simultaneously rotated such that when the locking teeth 756 of the engagement member 712 are aligned with the slots 766 between the locking teeth 768 of the base member 716 (and the locking teeth 768 are aligned with the slots 754 between the locking teeth 756), the locking teeth 756 of the engagement member 712 can slide into the slots 766 between the locking teeth 768 of the base member 716. Additional displacement of the engagement member 712 toward the base member 716 can push the locking teeth 756 farther into the slots 766 until the tooth lands 758 are in contact with the bottom of the slots 766. In some examples, the slots 766, 754 can be wedge-shaped slots (wedge-shaped slot 754 is shown in FIG. 29C; wedge-shaped slot 766 can be similar to wedge-shaped slot 754) to form a secure interlock between the sets of teeth 756, 768.

The base member 716 can include a proximal flange 762 that can be attached to the housing 720 to rotationally fix the base member 716 relative to the housing 720 and about the longitudinal axis L8. The output shaft 324 can extend proximally from the engagement member 712 and through central openings formed in the proximal flange 762 and base body 760. A gear 344 of a gear train (for example, gear train 308 shown in FIGS. 10A-10C) is coupled to the output shaft 346. The output shaft 324 can be axially aligned with the longitudinal axis L8. A spring 724 can be disposed around the output shaft 324 and between the engagement member 712 and the base member 716. In the free state, the spring 724 can apply a force to the engagement member 712 that biases the engagement member 712 towards the driver member 708.

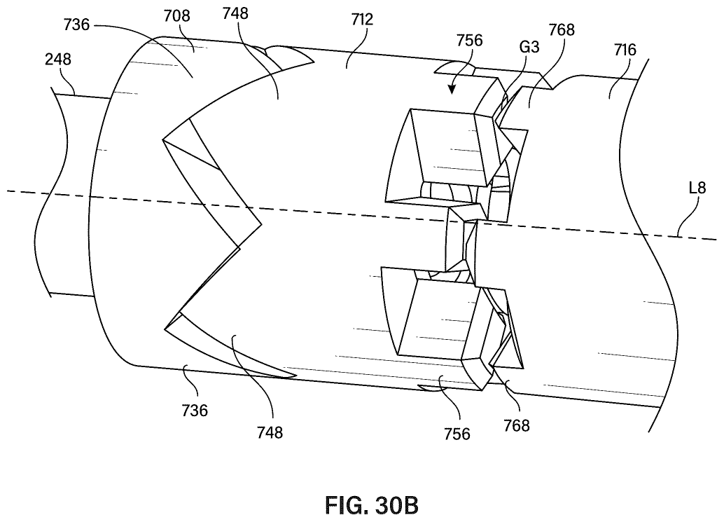

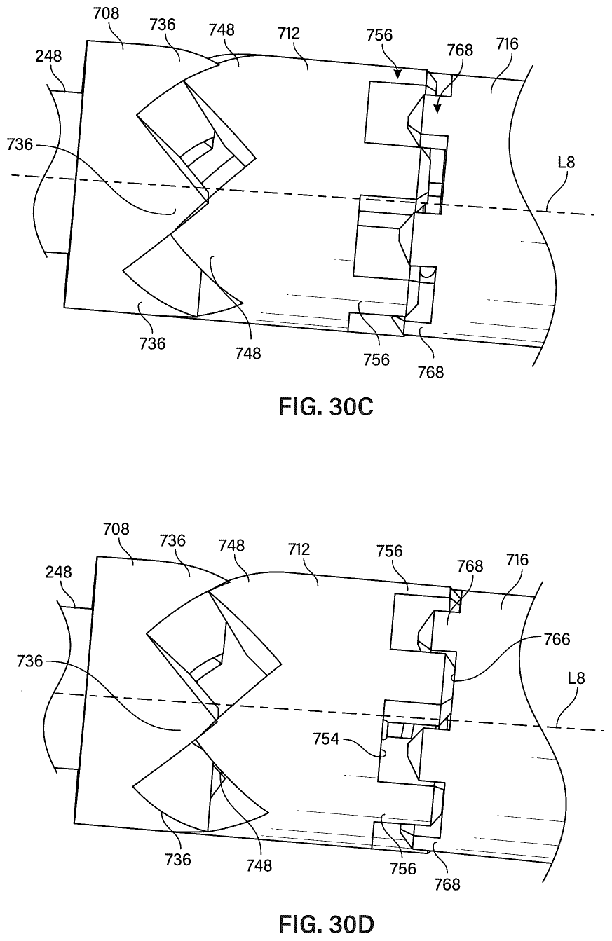

FIG. 30A shows an initial state of the torque limiter 700 where the torque on the actuator is below the threshold. In this initial state, the spring 724 is in a free state, the engagement member 712 is biased against the driver member 708, and the driver teeth 732 and engagement teeth 748 are fully engaged. In this state, the locking teeth 756 of the engagement member 712 are separated (axially spaced) from the locking teeth 768 of the base member 716 by a gap G2, which would allow the engagement member 712 to be rotatable by the output shaft 346 (shown in FIG. 29A). Since the driver teeth 732 and engagement teeth 748 are engaged, rotation of the engagement member 712 can be transferred to the actuator driver 248.

As the torque on the actuator driver 248 reaches the threshold, the engagement member 712 starts to move in the proximal direction (that is, in a direction towards the base member 716), as illustrated in FIG. 30B (where the gap G3 between the locking teeth 756, 768 is smaller than the previous gap G2 shown in FIG. 30A). The engagement teeth 748 start to slide over the driver teeth 736 during the proximal movement of the engagement member 712, causing the engagement member 712 to act against the spring 724. While the engagement member 712 is displaced towards the base member 716 but the set of locking teeth 756 of the engagement member 712 has not engaged the set of locking teeth 768 of the base member 716, rotation of the actuator driver 248 in either direction is possible by sliding of the engagement teeth 748 over the driver teeth 736 in either direction.

As the torque on the actuator driver 248 increases, further movement of the engagement member 712 towards the base member 716 causes the locking teeth 756 of the engagement member 712 to interlock with the locking teeth 768 of the base member 716, as illustrated in FIGS. 30C and 30D. Once the locking teeth 756, 768 are interlocked, rotation of the engagement member 712 will no longer be possible. As illustrated in FIG. 30D, the distance between the driver member 708, the engagement member 712, and the base member 716 can be designed such that the engagement teeth 748 can remain in contact with the driver teeth 732 even when the locking teeth 756, 768 are interlocked, thus effectively preventing rotational movement of the driver member 708 and actuator driver 248.

The torque limiter 600 and 700 can be accommodated within the handle of the delivery apparatus in the same manner described for the torque limiter 400. For example, either of the torque limiters 600 and 700 can replace the torque limiter 400 shown within the handle 204 in FIG. 21A. One or more of the actuator drivers 248 coupled to an output shaft 324 of the gearbox 300 can have a corresponding torque limiter 600 or 700. In some examples, each of the actuator drivers 248 can have a corresponding torque limiter 600 or 700 such that when a torque on any one of the actuator drivers 248 exceeds a threshold, the torque limiter 600 or 700 can act to halt the gearbox 300. By stopping the gearbox 300, damage to the native anatomy and/or the prosthetic heart valve can be prevented.

In some examples, while a user is rotating the knob (for example, the first knob 264 shown in FIG. 8) coupled to the gearbox 300 in a direction to expand the prosthetic heart valve, the torque on any one of the actuator drivers 248 may exceed the threshold. If the torque exceeds the threshold, the torque limiter (for example, torque limiter 600 or 700) coupled to the actuator driver 248 can act to halt the gearbox 300. When the gearbox 300 is halted, the user will no longer be able to rotate the knob in the direction to expand the prosthetic heart valve. At this point, if the prosthetic heart valve has already been expanded to the desired working diameter, the user can release the actuator drivers 248 from the prosthetic heart valve. Alternatively, if the prosthetic heart valve is not at the desired working diameter, the user can compress the prosthetic heart valve by rotating the knob in a direction opposite to the expansion direction. The user can remove the prosthetic heart valve from the implantation site.

Any of the systems, devices, apparatuses, etc. herein can be sterilized (for example, with heat, radiation, and/or chemicals, etc.) to ensure they are safe for use with patients, and any of the methods herein can include sterilization of the associated system, device, apparatus, etc. as one of the steps of the method. Examples of radiation for use in sterilization include, without limitation, gamma radiation and ultra-violet radiation. Examples of chemicals for use in sterilization include, without limitation, ethylene oxide and hydrogen peroxide.

The treatment techniques, methods, steps, etc. described or suggested herein or in references incorporated herein can be performed on a living animal or on a non-living simulation, such as on a cadaver, cadaver heart, anthropomorphic ghost, simulator (for example, with the body parts, tissue, etc. being simulated), etc.

### ADDITIONAL EXAMPLES

Additional examples based on principles described herein are enumerated below. Further examples falling within the scope of the subject can be configured by, for example, taking one feature of an example in isolation, taking more than one feature of an example in combination, or combining one or more features of one example with one or more features of one or more other examples.
Example 1. A delivery apparatus for a prosthetic heart valve comprises a handle having a proximal end, a distal end, and a cavity extending from the proximal end to the distal end; a first actuator driver having a proximal end portion disposed within the cavity and a distal end portion extending out of the cavity; a second actuator driver having a proximal end portion disposed within the cavity and a distal end portion extending out of the cavity; and a gear train disposed within the cavity and coupled to the proximal end portions of the first and second actuator drivers, the gear train configured to simultaneously rotate the first and second actuator drivers in opposite directions.
Example 2. A delivery apparatus according to Example 1, wherein the gear train comprises an input gear; a transmission gear engaged with and driven by the input gear; a first driving gear rotatably coupled to the transmission gear; a second driving gear engaged with and driven by the first driving gear; a first output gear engaged with and driven by the first driving gear, wherein the first actuator driver is coupled to the first output gear; and a second output gear engaged with and driven by the second driving gear, wherein the second actuator driver is coupled to the second output gear.
Example 3. A delivery apparatus according to Example 2, wherein the handle further comprises a rotatable knob coupled to the input gear.
Example 4. A delivery apparatus according to Example 3, wherein the rotatable knob is disposed at the proximal end of the handle.
Example 5. A delivery apparatus according to any one of Examples 3-4, wherein the input gear is coupled to an input shaft, and wherein the rotatable knob is coupled to the input shaft.
Example 6. A delivery apparatus according to Example 5, wherein the transmission gear and the first driving gear are coupled to a first shaft arranged in parallel to the input shaft, and wherein the second driving gear is coupled to a second shaft arranged in parallel to the first shaft.
Example 7. A delivery apparatus according to any one of Examples 5-6, wherein the handle has a first longitudinal axis extending from the proximal end to the distal end, and wherein the input shaft has a second longitudinal axis aligned with the first longitudinal axis.
Example 8. A delivery apparatus for a prosthetic heart valve comprises a handle having a longitudinal axis and a cavity extending along the longitudinal axis; a set of first actuator drivers, each first actuator driver having a proximal end portion disposed within the cavity and a distal end portion extending out of the cavity; a set of second actuator drivers, each second actuator driver having a proximal end portion disposed within the cavity and a distal end portion extending out of the cavity; a first driving gear coupled to the first actuation drivers, the first driving gear configured to rotate the first actuator drivers in a first direction; and a second driving gear coupled to the second actuator drivers, the second driving gear configured to rotate the second actuator drivers in a second direction that is opposite to the first direction.
Example 9. A delivery apparatus according to Example 8 further comprises an input shaft aligned with the longitudinal axis; an input gear coupled to the input shaft; a first shaft in parallel arrangement with the input shaft; and a transmission gear coupled to the first shaft and engaged with the input gear; wherein the first driving gear is coupled to the first shaft; and wherein the second driving gear is engaged with the first driving gear.
Example 10. A delivery apparatus according to Example 9 further comprises a second shaft in parallel arrangement with the first shaft; wherein the second driving gear is coupled to the second shaft.
Example 11. A delivery apparatus according to any one of Examples 8-10 further comprises a set of first output gears engaged with the first driving gear; and a set of second output gears engaged with the second driving gear; wherein each of the first output gears is coupled to the proximal end portion of one of the first actuator drivers; and wherein each of the second output gears is coupled to the proximal end portion of one of the second actuator drivers.
Example 12. A delivery apparatus according to any one of Examples 9-11, wherein the handle comprises a rotatable knob coupled to the input shaft.
Example 13. A delivery apparatus according to any one of Examples 7-12 further comprises a shaft assembly coupled to the handle, the shaft assembly comprising a first delivery shaft having a first lumen and a second delivery shaft having a plurality of lumens, the second delivery shaft extending through the first lumen, wherein the first and second actuator drivers extend through the plurality of lumens of the second delivery shaft.
Example 14. A delivery apparatus according to any one of Examples 7-13, wherein the set of first actuator drivers comprises three first actuator drivers, and wherein the set of second actuator drivers comprises three second actuator drivers.
Example 15. A prosthetic heart valve comprises a frame having an inflow end, an outflow end, and a longitudinal axis extending from the inflow end to the outflow end, the frame movable between a radially expanded configuration and a radially compressed configuration; a first actuator coupled to the frame at a first position; and a second actuator coupled to the frame at a second position that is spaced from the first position along a circumference of the frame; wherein rotation of the first actuator in a first rotational direction and rotation of the second actuator in a second rotational direction, opposite the first rotational direction, moves the frame between the radially expanded configuration and the radially compressed configuration.
Example 16. A prosthetic heart valve according to Example 15, wherein the first actuator comprises a threaded portion having a first configuration, and wherein the second actuator comprises a threaded portion having a second configuration that is opposite to the first configuration.
Example 17. A prosthetic heart valve according to any one of Examples 15-16, wherein the frame comprises a plurality of support posts aligned with the longitudinal axis; and a plurality of struts interconnecting the support posts; wherein the first actuator is coupled to a first support post of the plurality of support posts; and wherein the second actuator is coupled to a second support post of the plurality of support posts.
Example 18. A prosthetic heart valve according to Example 17, wherein each of the first support post and the second support post comprises a gap, wherein each of the actuators comprises an actuator rod extending across the gap in the respective support post, and wherein rotation of each of the actuation rods adjusts a size of the gap in the respective support post.
Example 19. A prosthetic heart valve according to any one of Examples 15-18 further comprises a valvular structure disposed within and coupled to the frame.
Example 20. A delivery assembly comprises a prosthetic heart valve, which comprises a frame having an inflow end, an outflow end, and a longitudinal axis extending from the inflow end to the outflow end, the frame movable between a radially expanded configuration and a radially compressed configuration; a first actuator coupled to the frame at a first position; and a second actuator coupled to the frame at a second position that is spaced from the first position along a circumference of the frame. The delivery assembly further comprises a handle having a proximal end, a distal end, and a cavity extending from the proximal end to the distal end; a first actuator driver having a proximal end portion disposed within the cavity and a distal end portion extending out of the cavity and releasably coupled to the first actuator; a second actuator driver having a proximal end portion disposed within the cavity and a distal end portion extending out of the cavity and releasably coupled to the second actuator; and a gear train disposed within the cavity and coupled to the proximal end portions of the first and second actuator drivers, the gear train configured to simultaneously rotate the first and second actuator drivers in opposite directions.
Example 21. The delivery assembly according to Example 20, wherein the prosthetic heart valve further comprises a valvular structure disposed within and coupled to the frame.
Example 22. A delivery apparatus for a prosthetic heart valve comprises a handle having a cavity; a gearbox disposed within the cavity, the gearbox comprising at least one output shaft and a gear coupled thereto; an actuator driver having a predefined torque limit range; and a rotatable assembly coupling the at least one output shaft to the actuator driver, the rotatable assembly having a first rotational state wherein the at least one output shaft and the actuator driver rotate together about a longitudinal axis and a second rotational state wherein the at least one output shaft and the actuator driver do not rotate together about the longitudinal axis, wherein the first rotational state corresponds to when a torque applied to the actuator driver is below the predefined torque limit range, and wherein the second rotational state corresponds to when the torque applied to the actuator driver is within the predefined torque limit range.
Example 23. A delivery apparatus according to Example 22, wherein the rotatable assembly comprises a first rotatable body coupled to the at least one output shaft, a second rotatable body coupled to the actuator driver, and a rotational bias member coupling the first rotatable body to the second rotatable body.
Example 24. A delivery apparatus according to Example 23, wherein the rotational bias member comprises a torsion spring having a coil portion, a first end portion coupled to the first rotatable body, and a second end portion coupled to the second rotatable body.
Example 25. A delivery apparatus according to Example 24, wherein the torsion spring is configured to twist in a direction to decrease an angular spacing between the first and second end portions when the torque applied to the actuator driver is within predefined torque limit range.
Example 26. A delivery apparatus according to Example 24, wherein the torsion spring has a preload, and wherein the rotatable assembly transitions from the first rotational state to the second rotational state when the torque applied to the actuator driver exceeds the preload in the torsion spring.
Example 27. A delivery apparatus according to any one of Examples 24-26, further comprising a connector shaft extending through the second rotatable body, wherein the coil portion is disposed around the connector shaft.
Example 28. A delivery apparatus according to Example 27, wherein a first end portion of the connector shaft extends into the first rotatable body and a second end portion of the connector shaft is coupled to the actuator driver.
Example 29. A delivery apparatus according to any one of Example 23-28, wherein the rotatable assembly further comprises a tapered channel and a wedge member movably disposed within the tapered channel, and wherein the wedge member prevents rotational movement of the first and second rotatable bodies when the wedge member is positioned at a predetermined location within the tapered channel.
Example 30. A delivery apparatus according to Example 29, wherein the wedge member has a first end portion coupled to the first rotatable body and a second end portion disposed in the tapered channel.
Example 31. A delivery apparatus according to any one of Examples 29-30, wherein the tapered channel is formed on a periphery of the second rotatable body.
Example 32. A delivery apparatus according to Example 31, wherein the rotatable assembly further comprises a housing, wherein the tapered channel is formed between an inner surface of the housing and an outer surface of the second rotatable body, and wherein the wedge member interferingly engages with both the inner and outer surfaces at the predetermined location.
Example 33. A delivery apparatus according to Example 32, wherein the outer surface of the second rotatable body comprises a recessed portion, and wherein the tapered channel is formed between the recessed portion and the inner surface of the housing.
Example 34. A delivery apparatus according to Example 33, wherein the outer surface of the second rotatable body comprises a first radial shoulder and a second radial shoulder circumferentially spaced around the second rotatable body, and wherein the recessed portion is formed between the first and second radial shoulders.
Example 35. A delivery apparatus according to any one of Examples 32-34, wherein the housing is a compartment of a gearbox housing.
Example 36. A delivery apparatus according to Example 25, wherein the second rotatable body comprises a pair of tapered recessed portions at diametrically opposed positions, the pair of tapered recessed portions defining a pair of tapered channels.
Example 37. A delivery apparatus according to Example 36, further comprising a pair of wedge members, each of the wedge members having a first end portion coupled to the first rotatable body and a second end portion disposed in one of the tapered channels, each of the wedge members movable along the respective tapered channel in response to relative movement between the first and second rotatable bodies during twisting of the torsion spring, the second end portion of each wedge member configured to form a wedge at a predetermined location within the respective tapered channel that prevents further rotation of the first and second rotatable bodies.
Example 38. A delivery apparatus according to any one of Examples 36-37, wherein each of the tapered channels is tapered in a direction along a circumference of the second rotatable body.
Example 39. A delivery apparatus according to any one of Examples 24-38, wherein the first rotatable body comprises a first recess receiving a first portion of the coil portion of the torsion spring and a first slot receiving the first end portion of the torsion spring, and wherein the second rotatable body comprises a second recess receiving a second end portion of the coil portion and a second slot receiving the second end portion.
Example 40. A delivery apparatus according to Example 39, wherein the actuator driver extends through the coil portion and the first and second recesses.
Example 41. A delivery apparatus for a prosthetic heart valve comprises a handle having a cavity; a gearbox disposed within the cavity, the gearbox comprising a plurality of output shafts and a plurality of output gears coupled thereto; a plurality of actuator drivers, each actuator driver having a predefined torque limit range; and a plurality of rotatable assemblies, each of the rotatable assemblies coupled at a first end to one of the output shafts and at a second end to one of the actuator drivers. Each rotatable assembly comprises a first rotatable body; a second rotatable body; and a rotational biasing member coupling the first rotatable body to the second rotatably body; wherein the rotational biasing member biases the first rotatable body and the second rotatable body to a position in which the first rotatable body and the second rotatable body rotate together about a longitudinal axis when a torque applied to the actuator driver is below the predefined torque limit range; and wherein the rotational biasing member permits relative rotation between the first rotatable body and the second rotatable body about the longitudinal axis when the torque applied to the actuator driver is within the predefined torque limit range.
Example 42. A delivery apparatus for a prosthetic heart valve comprises a handle body having a longitudinal axis; and a gearbox pivotably mounted within the handle body and about the longitudinal axis.
Example 43. A delivery apparatus according to Example 42, wherein the gearbox comprises a gearbox housing and a gear train disposed within the gearbox housing, and further comprising a rotatable knob coupled to the gear train.
Example 44. A delivery apparatus for a prosthetic heart valve comprises a handle body having a longitudinal axis; a gearbox pivotable mounted within the handle body and about the longitudinal axis; and a stop member coupled to the handle body and positioned to limit pivoting of the gearbox about the longitudinal axis when the gearbox is pivoted in a predetermined direction.
Example 45. A delivery apparatus according to Example 44, wherein the gearbox comprises a gear train having an input shaft aligned with the longitudinal axis; a gearbox housing enclosing the gear train, the gearbox housing having an extension arm projecting from an outer surface of the gearbox housing and a protrusion member attached to the extension arm, wherein the protrusion member is configured to contact the stop member when the gearbox is rotated in the predetermined direction.
Example 46. The delivery apparatus according to Example 45, wherein the protrusion member is oriented in a direction transverse to the longitudinal axis.
Example 47. The delivery apparatus according to any one of Examples 44-46, wherein the predetermined direction is in a direction to expand the prosthetic heart valve.
Example 48. The delivery apparatus according to any one of Examples 44-47, wherein the stop member comprises a load cell, and wherein the protrusion member is configured to apply load to the load cell when the gearbox is pivoted in the predetermined direction.
Example 49. A delivery apparatus for a prosthetic heart valve comprises a handle body having a longitudinal axis; a load cell coupled to the handle body, the load cell having a first axial axis positioned tangentially to a circular path centered around the longitudinal axis; and a gearbox pivotably mounted about the longitudinal axis, the gearbox having a protrusion member with a first axial axis positioned tangentially to the circular path, the protrusion member configured to contact the load cell as the gearbox is pivoted in a predetermined direction corresponding to operation of the gearbox to expand the prosthetic heart valve.
Example 50. A delivery apparatus according to Example 49, wherein the gearbox comprises a gearbox housing and a gear train disposed within the gearbox housing, and further comprising a rotatable knob coupled to the gear train.
Example 51: A delivery apparatus for a prosthetic heart valve comprises an actuator driver; a gearbox comprising at least one output shaft; an engagement member coupled to the at least one output shaft and rotatable about a longitudinal axis with the at least one output shaft, the engagement member having a first engagement surface and a first locking surface spaced apart along the longitudinal axis; a driver member coupled to the actuator driver and rotatable about the longitudinal axis, the driver member having a second engagement surface in opposing relation to the first engagement surface and engaged with the first engagement surface; and a base member rotationally fixed relative to the longitudinal axis, the base member having a second locking surface in opposing relation to the first locking surface; wherein the engagement member is axially displaceable along the longitudinal axis in response to a torque on the actuator driver and between a first position in which the first locking surface is separated from the second locking surface and a second position in which the first locking surface is interlocked with the second locking surface, and wherein the second position corresponds to a state in which the torque on the actuator driver exceeds a threshold.
Example 52: The delivery apparatus according to any example herein, particularly Example 51, wherein the at least one output shaft extends through a central opening of the base member.
Example 53: The delivery apparatus according to any example herein, particularly any one of Examples 51 to 52, further comprising a spring arranged to apply a force to the engagement member that biases the first engagement surface against the second engagement surface in the first position.
Example 54: The delivery apparatus according to any example herein, particularly Example 53, wherein the spring is disposed around the at least one output shaft and between the engagement member and the base member.
Example 55: The delivery apparatus according to any example herein, particularly any one of Examples 51 to 54, wherein the first locking surface comprises a first set of locking teeth, and wherein the second locking surface comprises a second set of locking teeth complementary to the first set of locking teeth.
Example 56: The delivery apparatus according to any example herein, particularly Example 55, wherein the first set of locking teeth comprises a plurality of first teeth separated by first slots, wherein the second set of locking teeth comprises a plurality of second teeth separated by second slots, wherein the plurality of first teeth are configured to extend into the second slots and the plurality of second teeth are configured to extend into the first slots to interlock the first set of locking teeth with the second set of locking set.
Example 57: The delivery apparatus according to any example herein, particularly any one of Examples 51 to 56, wherein the first engagement surface comprises a set of engagement teeth, and wherein the second engagement surface comprises a set of driver teeth complementary to the set of engagement teeth.
Example 58: The delivery apparatus according to any example herein, particularly Example 57, wherein each tooth of the set of engagement teeth comprises a first axial tooth surface and a first inclined tooth surface joined at a first tooth apex; wherein each tooth of the set of driver teeth comprises a second axial tooth surface and a second inclined tooth surface joined at a second tooth apex; and wherein the first inclined tooth surfaces of the set of engagement teeth slide along the second inclined tooth surfaces of the set of engagement teeth during axial displacement of the engagement member.
Example 59: The delivery apparatus according to any example herein, particularly Example 57, wherein each tooth of the set of engagement teeth comprises a first inclined tooth surface and a second inclined tooth surface joined at a first tooth apex; wherein each tooth of the set of driver teeth comprises a third inclined tooth surface and a fourth inclined tooth surface joined at a second tooth apex; and wherein the first and second inclined tooth surfaces slide over the third and fourth inclined tooth surfaces in a first rotational direction about the longitudinal axis or a second rotational direction about the longitudinal axis during axial displacement of the engagement member.
Example 60: The delivery apparatus according to any example herein, particularly any one of Examples 51 to 59, further comprising a housing, wherein the engagement member, the driver member, and the base member are disposed inside the housing.
Example 61: The delivery apparatus according to any example herein, particularly Example 60, wherein the engagement member and the driver member are rotatable relative to the housing, and wherein the base member is fixedly coupled to the housing.
Example 62: The delivery apparatus according to any example herein, particularly any one of Examples 60-61, wherein the housing is coupled to the gearbox.
Example 63: The delivery apparatus according to any example herein, particularly any one of Examples 51 to 62, further comprising a handle, wherein the gearbox is disposed in a cavity within the handle.
Example 64: A delivery apparatus for a prosthetic heart valve comprises a handle having a cavity; a gearbox disposed within the cavity, the gearbox comprising at least one output shaft; an actuator driver extending into the cavity; and a torque limiter coupling the actuator driver to the at least one output shaft, the torque limiter comprising: an engagement member coupled to the at least one output shaft and rotatable about a longitudinal axis in response to rotation of the at least one output shaft, the engagement member comprising a set of engagement teeth at a first end and a first set of locking teeth at a second end spaced from the first end; a driver member rotatable about the longitudinal axis and coupled to the actuator driver, the driver member comprising a set of driver teeth in opposing relation to the set of engagement teeth and slidably engaged with the set of engagement teeth; a base member rotationally fixed relative to the longitudinal axis, the base member comprising a second set of locking teeth in opposing relation to the first set of locking set; and wherein the engagement member is axially displaceable along the longitudinal axis in response to a torque on the actuator driver, and wherein the engagement member is axially displaceable to engage the first set of locking teeth with the second set of locking teeth when the torque on the actuator driver exceeds a threshold.
Example 65: The delivery apparatus according to any example herein, particularly Example 64, wherein the gearbox comprises a plurality of output shafts; and wherein a plurality of torque limiters couples the plurality of output shafts to a corresponding plurality of actuator drivers.
Example 66: A delivery apparatus for a prosthetic heart valve comprises an actuator driver; a gearbox comprising at least one output shaft; a base member rotationally fixed relative to a longitudinal axis; and an engagement member movably coupled to the actuator driver and fixedly coupled to the at least one output shaft, the engagement member axially displaceable along the longitudinal axis and relative to the actuator driver and the base member in response to a torque on the actuator driver, wherein the engagement member is configured to engage the base member when the torque on the actuator driver exceeds a threshold.
Example 67: A method comprises coupling a prosthetic heart valve to at least one actuator driver of a delivery apparatus, wherein an engagement member is movably coupled to the at least one actuator driver and fixedly coupled to an output shaft of a gearbox of the delivery apparatus, wherein the engagement member is axially displaceable along a longitudinal axis and between the at least one actuator driver and a base member that is rotationally fixed relative to the longitudinal axis in response to a torque on the at least one actuator driver; and rotating the output shaft of the gearbox to rotate the at least one actuator driver in a first direction to radially expand the prosthetic heart valve to a working diameter, wherein rotation of the output shaft automatically stops by engagement of the engagement member with the base member when the torque on the at least one actuator driver exceeds a threshold.
Example 68: The method of any example herein, particularly Example 67, further comprises inserting the prosthetic heart valve and a distal end of the delivery apparatus into a patient's vasculature; and advancing the delivery apparatus through the patient's vasculature to position the prosthetic heart valve at an implantation site.
Example 69: The method according to any example herein, particularly any one of Examples 67-68, further comprises rotating the output shaft in a second direction to radially compress the prosthetic heart valve.
Example 70: The method according to any example herein, particularly any one of Examples 67-69, further comprises releasing the prosthetic heart valve from the at least one actuator driver.
Example 71: The method according to any example herein, particularly any one of Examples 67-70, wherein rotating the output shaft comprises rotating a knob coupled to the gearbox.
Example 72: A method comprising sterilizing any one of the delivery apparatus according to any one of Examples 1-14 and 20-66.
Example 73: A method comprising sterilizing any one of the prosthetic heart valves according to any one of Examples 15-19.
Example 74: A method comprising implanting a prosthetic device using any one of the delivery apparatus according to any one of claims 1-14 and 20-66.
Example 75: A method of simulating an implantation procedure for a prosthetic device using any one of the delivery apparatus according to any one of Examples 1-14 and 20-66.
Example 76: A delivery apparatus for a prosthetic heart valve comprises a driver member rotatable about a first axis; an output shaft rotatable about the first axis; an engagement member rotatable coupled to the output shaft, the engagement member configured to move between an unlocked state and a locked state; wherein the driver member, when rotating about the first axis, is configured to generate torque on the engagement member, wherein the torque on the engagement member is configured to cause the engagement member to move from the unlocked state to the locked state when the torque on the engagement member exceeds a predetermined threshold amount, and wherein the engagement member, when in the locked state, prevents the output shaft from rotating about the first axis.

The subject matter has been described with a selection of implementations and examples, but these preferred implementations and examples are not to be taken as limiting the scope of the subject matter since many other implementations and examples are possible that fall within the scope of the subject matter. The scope of the claimed subject matter is defined by the claims.

1. A delivery apparatus for a prosthetic heart valve, comprising:
a handle having a longitudinal axis and a cavity extending along the longitudinal axis;
a set of actuator drivers comprising a first actuator driver and a second actuator driver, the first actuator driver and the second actuator driver each having proximal end portions disposed within the cavity and distal end portions extending out of the cavity; and
a gear train coupled to the proximal end portions of the first actuation driver and the second actuator driver, the gear train configured to simultaneously rotate the first actuation driver and second actuator driver in opposite directions.

2. The delivery apparatus of claim 1, wherein the gear train comprises an input gear, and further comprising:
an input shaft disposed within the cavity and coupled to the input gear; and
a rotatable knob disposed at a proximal end of the handle and coupled to the input shaft.

3. The delivery apparatus of claim 2, wherein the gear train further comprises:
a transmission gear engaged with and driven by the input gear;
a first driving gear rotatably coupled to the transmission gear;
a second driving gear engaged with and driven by the first driving gear;
a first output gear engaged with and driven by the first driving gear, wherein the first actuator driver is coupled to the first output gear; and
a second output gear engaged with and driven by the second driving gear, wherein the second actuator driver is coupled to the second output gear.

4. The delivery apparatus of claim 1, wherein the first actuator driver is one of a plurality of first actuator drivers, wherein the second actuator driver is one of a plurality of second actuator drivers, and wherein the gear train is configured to simultaneously rotate the plurality of first actuator drivers in a first direction and the plurality of second actuator drivers in a second direction which is opposite to the first direction.

5. The delivery apparatus of claim 4, wherein the gear train comprises a first driving gear coupled to the plurality of first actuator drivers and configured to rotate the plurality of first actuator drivers in the first direction and a second driving gear coupled to the plurality of second actuator drivers and configured to rotate the plurality of second actuator drivers in the second direction.

6. The delivery apparatus of claim 5, wherein the gear train further comprises:
a plurality of first output gears engaged with the first driving gear; and
a plurality of second output gears engaged with the second driving gear;
wherein each of the first output gears is coupled to the proximal end portion of one of the first actuator drivers; and
wherein each of the second output gears is coupled to the proximal end portion of one of the second actuator drivers.

7. The delivery apparatus of claim 1, further comprising a shaft assembly coupled to the handle, the shaft assembly comprising a first delivery shaft having a first lumen and a second delivery shaft having a plurality of lumens, the second delivery shaft extending through the first lumen, wherein the first and second actuator drivers extend through the plurality of lumens of the second delivery shaft.

8. The delivery apparatus of claim 1, further comprising a gearbox mounted within the cavity and pivotable about the longitudinal axis, wherein the gearbox houses one or more components of the gear train.

9. The delivery apparatus of claim 8, further comprising a stop member coupled to the handle and positioned to limit pivoting of the gearbox about the longitudinal axis when the gearbox is pivoted in a predetermined direction.

10. The delivery apparatus of claim 9, wherein the stop member comprises a load cell, and wherein the gearbox comprises a protrusion member configured to apply load to the load cell when the gearbox is pivoted in the predetermined direction.

11. A delivery apparatus for a prosthetic heart valve, comprising:
a driver member rotatable about a first axis;
an output shaft rotatable about the first axis; and
an engagement member rotatably coupled to the output shaft, the engagement member configured to move between an unlocked state and a locked state;
wherein the driver member, when rotating about the first axis, is configured to generate torque on the engagement member, wherein the torque on the engagement member is configured to cause the engagement member to move from the unlocked state to the locked state when the torque on the engagement member exceeds a predetermined threshold amount, and wherein the engagement member, when in the locked state, prevents the output shaft from rotating about the first axis.

12. The delivery apparatus of claim 11, wherein the engagement member is axially displaceable along the first axis and relative to the driver member in response to the torque, wherein the engagement member comprises a set of engagement teeth at a first end, and wherein the driver member comprises a set of driver teeth in opposing relation to the set of engagement teeth.

13. The delivery apparatus of claim 12, further comprising a bias member arranged to apply a bias force to the engagement member that biases the set of engagement teeth against the set of driver teeth in the unlocked state.

14. The delivery apparatus of claim 13, wherein each tooth of the set of engagement teeth comprises a first axial tooth surface and a first inclined tooth surface joined at a first tooth apex;
wherein each tooth of the set of driver teeth comprises a second axial tooth surface and a second inclined tooth surface joined at a second tooth apex; and
wherein the first inclined tooth surfaces of the set of engagement teeth slide along the second inclined tooth surfaces of the set of engagement teeth during axial displacement of the engagement member.

15. The delivery apparatus of claim 13, wherein each tooth of the set of engagement teeth comprises a first inclined tooth surface and a second inclined tooth surface joined at a first tooth apex;
wherein each tooth of the set of driver teeth comprises a third inclined tooth surface and a fourth inclined tooth surface joined at a second tooth apex; and
wherein the first and second inclined tooth surfaces slide over the third and fourth inclined tooth surfaces in a first rotational direction about the first axis or a second rotational direction about the first axis during axial displacement of the engagement member along the first axis.

16. The delivery apparatus of claim 13, further comprising a base member rotationally fixed relative to the first axis, wherein the engagement member is axially displaceable along the first axis to engage the base member when the torque on the engagement member exceeds the predetermined threshold amount.

17. The delivery apparatus of claim 16, wherein:
the engagement member comprises a first set of locking teeth at a second end spaced from the first end; and
the base member comprises a second set of locking teeth in opposing relation to the first set of locking teeth, the second set of locking teeth configured to engage with the first set of locking teeth in the locked state.

18. The delivery apparatus of claim 16, wherein the output shaft extends through a central opening of the base member, and wherein the bias member comprises a spring disposed around the output shaft and between the engagement member and the base member.

19. A method comprising:
coupling a prosthetic heart valve to a driver member configured to generate torque on an engagement member, wherein the engagement member is coupled to an output shaft of a gearbox and has a locked state and an unlocked state;
rotating the output shaft in a first direction to radially expand the prosthetic heart valve to a working diameter;
transferring rotation of the output shaft to the driver member through engagement of the engagement member with the driver member in the unlocked state of the engagement member; and
causing the engagement member to move from the unlocked state to the locked state when the torque on the engagement member exceeds a predetermined threshold amount.

20. The method of claim 19, wherein rotating the output shaft comprises rotating a knob coupled to the gearbox.
